(19) United States
(12) Patent Application Publication
(10) Pub. No.: US 2008/0125388 A1
(43) Pub. Date: May 29, 2008

(54) Methods and compositions for CPG15-2

(76) Inventors: Elly Nedivi, Brookline, MA (US); Tadahiro Fujino, Somerville, MA (US)

Correspondence Address: CLARK & ELBING LLP, 101 FEDERAL STREET, BOSTON, MA, 02110, US

(21) Appl. No.: 11/708,402

(22) Filed: Feb. 20, 2007

Related U.S. Application Data
(62) Division of application No. 10/955,205, filed on Sep. 30, 2004, now abandoned.
(60) Provisional application No. 60/507,359, filed on Sep. 30, 2003.

(51) Int. Cl. A61K 31/70; C12N 5/02

(52) U.S. Cl. 514/44; 435/377

(57) ABSTRACT

Disclosed herein are compositions of CPG 15-2 and methods for treating conditions of excessive cell death, such as neurological conditions, using such compositions. Compounds that inhibit the activity of CPG 15-2 are also disclosed herein for the treatment of conditions of undesirable cell survival, such as cancer.

### CROSS REFERENCES TO RELATED APPLICATIONS

This application is a divisional of U.S. patent application Ser. No. 10/955,205 filed on Sep. 30, 2004, which in turn claims the benefit of the filing date of U.S. Provisional Application No. 60/507,359, filed on Sep. 30, 2003, herein incorporated by reference.

### STATEMENT AS TO FEDERALLY SPONSORED RESEARCH

This invention was made in part with support from the Government through NIH Grant No. 5-R01-EY11894. The Government has certain rights in the invention.

### BACKGROUND OF THE INVENTION

The invention relates to compositions of CPG 15-2 and methods of using CPG 15-2 to treat various conditions, including neurological conditions.

Neurogenesis is an adaptive process whereby a large and excessive population of neurons are initially produced followed by a reduction in the number of neurons as a result of the presence or absence of stimuli from the target organ and the presence or absence of neurotrophic factors in the environment surrounding the neurons. The extensive neuronal remodeling that occurs in response to stimuli both during development and in the adult brain provides the foundation for learning and memory, as well as adaptive reorganization of primary sensory maps. In sum, the proper development of the mature vertebrate nervous systems requires a delicate balance of neuronal cell growth and death.

Many neurological conditions are the result of a shift in the balance towards increased and inappropriate neuronal cell death. For example, the increased death of hippocampal and cortical neurons is responsible for many of the symptoms of Alzheimer's disease (AD); the death of midbrain neurons underlies Parkinson's disease (PD); the death of neurons in the striatum contributes to Huntington's disease (HD); and the death of lower motor neurons results in amyotrophic lateral sclerosis (ALS). Many other neurological diseases and dysfunctions such as stroke, trauma, spinocerebellar ataxis, and peripheral neuropathies are also characterized by excessive cell death.

There are generally two mechanisms by which cell death can occur: apoptosis and necrosis. Necrosis is thought to follow traumatic injury and is characterized by cytoplasmic vacuolization and swelling of the cellular organelles. In necrotic cell death, the plasma membrane lyses, resulting in massive death of groups of cells throughout the affected tissue. Apoptosis, or programmed cell death, is an active process that proceeds via protein synthesis, nuclear fragmentation, chromosome condensation, and activation of proteolytic caspase cascades. Death of a cell by apoptotic pathways does not trigger the death of cells proximal to the apoptotic cell.

The apoptosis pathway is known to play a critical role in numerous normal and pathological events. For example, apoptosis is directly involved in embryonic development, viral pathogenesis, cancer, autoimmune conditions, and neurodegenerative diseases. There are many features of apoptotic cell death that are shared by a wide variety of cell types.

The types of cell death involved in specific neuropathologies varies and, in some cases, is difficult to classify as necrotic or apoptotic. This is not surprising, given that many neurodegenerative diseases are chronic progressive conditions with cell death occurring over a period of five to twenty years or more. In instances of chronic progressive conditions there exists a mixture of necrotic and apoptotic cell death which contributes to the disease progression over time. Even in the case of traumatic injury it is believed that after the initial insult necrotic cell death occurs, and that this necrosis actually triggers a secondary cascade of apoptotic cell death resulting in a more severe spread of cell damage and death than the damage caused by the initial traumatic injury itself.

Emphasis has been placed on understanding the key proteins or factors involved in regulating cell death, particularly apoptosis, in general, and specifically in neuronal cells. Many signals have been identified as initiators of apoptosis in neurons. Extracellular initiation signals include the absence of neurotrophic factors, such as nerve growth factor (NGF) or brain-derived neurotrophic factor (BDNF) in the surrounding environment, activation of a death receptor (e.g., TNF-R/FAS), increased oxidative stress, and the presence of metabolic or environmental toxins. Intracellular initiation signals include disruption of mitochondrial function and the release of mitochondrial factors such as cytochrome c. Following initiation of apoptosis, the activation step takes place. Activation includes proteolytic processing of caspases, an event which allows the caspases to trigger the final step in the process, the effector step. The effector step occurs via the maturation and activation of the effector caspases, again through proteolytic cleavage.

There are many signaling proteins that regulate activation of apoptotic pathways. Examples of these signaling proteins include B-cell lymphoma 2 (Bcl-2) family proteins, caspases (both upstream activator caspases and downstream effector caspases), telomerase, prostate apoptosis response 4 (Par 4), NFκB, inhibitors of apoptosis (IAPs), p53, and calcium-binding proteins, to name but a few. Individual proteins either function to promote or inhibit apoptosis. In addition, some proteins can function both to promote and inhibit apoptosis. It is the delicate balance between pro-apoptotic and anti-apoptotic factors that results in the dynamic events of neuronal development and remodeling.

A great deal of focus has been placed on identifying genes expressed in neurons that affect this balance between cell growth or survival, and cell death. cpg15 (candidate plasticity gene) was recently identified in a differential screen for genes upregulated by activity in adult hippocampus. CPG15 protein was found to be expressed in differentiated projection neurons within sensory systems throughout the brain, including the auditory system, olfactory system, and visual system. CPG15 is also expressed in the spinal cord and at lower levels outside the central nervous system. In the post-natal and adult brain, peak expression of CPG 15 occurs during periods of neuronal dendritic arbor growth and synaptogenesis. In the adult rat, CPG 15 is induced in the brain by kainate and in the visual cortex by light. The CPG 15 protein has an N-terminal secretion signal characteristic of extracellular proteins, a C-terminal domain comprised of hydrophobic residues indicative of a glycosyl-phosphatidylinositol (GPI) link to the cell surface, and six cysteine residues thought to be critical for correct protein folding. We have demonstrated that it is the soluble form of CPG 15 that functions to promote cell survival and to promote dendritic arbor growth and differentiation.

The role of specific signaling proteins in cell growth and cell death pathways has been studied intensively over the past few years and although several candidate therapeutic targets have been identified, cures for conditions, such as neurological conditions, that are associated with increased cell death, have remained elusive. Given the prevalence of neurological conditions such as AD, PD, HD, and ALS, as well as stroke and trauma, there exists a need for effective therapeutic agents that target the molecules that influence cell death, particularly neuronal cell death. In addition, given the commonalties in apoptotic pathways at the cellular levels, therapeutic agents that are effective for the treatment of neurological conditions are likely to be effective for the treatment of other cell death related conditions.

### SUMMARY OF THE INVENTION

We have discovered a novel gene, hereafter referred to as cpg15-2 and the protein encoded by this gene, hereafter referred to as CPG15-2. CPG15-2 has very little nucleotide or amino acid sequence homology to CPG15 and also appears to have a distinct tissue expression and developmental onset expression pattern from that of CPG15. Despite these distinctions, we have discovered that CPG15-2 shares both structural and functional homology with soluble CPG15. We have discovered that CPG 15-2 can act as a survival factor by rescuing hippocampal and cortical neurons from cell death. CPG15-2 can also act to promote growth and differentiation of cells.

Cell death mechanisms in hippocampal and cortical neurons follow classic programmed cell death signaling pathways that are common to additional types of neurons as well as other types of cells. Accordingly, we believe CPG15-2, can be used to promote cell survival in various types of cells where excess apoptosis contributes to disease pathology, including myocytes, liver cells, endothelial cells, hematopoietic cells, bone cells, and immune cells. Conversely, inhibitors of CPG15-2 can be used to promote cell death in various types of cells where excessive proliferation contributes to disease pathology, for example, cancers. Since CPG15-2 affects classic programmed cell death pathways, the present invention also includes the use of the cpg15-2 gene and CPG 15-2 protein as a tool for screening for interacting molecules that modulate cell death, cell survival, and cell differentiation pathways. Once identified, these molecules can then be used to promote cell survival where excessive cell death contributes to the pathologies of the disease or to promote cell death where cell survival and division contribute to the pathologies of the disease.

Accordingly, in a first aspect, the invention provides a substantially pure CPG15-2 protein, including a sequence substantially identical (e.g., 85%, 90%, 95%, 99% or greater) to the amino acid sequence of human CPG 15-2 (SEQ ID NO: 2) or mouse CPG 15-2 (SEQ ID NO: 4), preferably over the entire length of the sequence. In a preferred CPG 15-2 protein has at least 87% sequence identity to the amino acid sequence of SEQ ID NO: 2 or 4. In additional preferred embodiments, the CPG15-2 protein includes a sequence that is at least 85%, preferably 87%, 90%, 95%, or 100% identical to the core domain sequence set forth in SEQ ID NOs: 5 or 6; the amino acid sequences 35 to 131 of SEQ ID NO: 4; or amino acids 38 to 134 of SEQ ID NO: 2. Most preferably the CPG15-2 includes any one, two, three, four, five, or six of the following conserved cysteine residues: Cys 39, 49, 67, 76, 84, or 112 of SEQ ID NO: 4 or Cys 42, 52, 70, 79, 87, or 115 of SEQ ID NO: 2. In a preferred embodiment of the first aspect, the substantially pure CPG 15-2 protein includes the sequence of SEQ ID NOs: 2 or 4. CPG15-2 can also include a sequence substantially identical (e.g., 85%, 90%, 95%, 99% or greater) to amino acids 1 to 20 or 116 to 162 of SEQ ID NO: 4 or amino acids 1 to 40 or 118 to 165 of SEQ ID NO: 2. The CPG 15-2 protein can include any form such as the membrane bound form, the secreted soluble form, or the unprocessed form. The CPG15-2 can lack either a signal sequence or a GPI linkage sequence or both. In preferred embodiments, the CPG 15-2 is a soluble CPG 15-2 protein that lacks a signal sequence and a GPI linkage sequence and that has the in vitro biological activity of a CPG 15-2 protein wherein (a) the signal sequence and the GPI linkage sequence of the CPG15-2 protein have been cleaved; (b) the CPG 15-2 protein has been bound to a cell membrane; and (c) the CPG15-2 protein has been released from the cell. The CPG15-2 compound of the invention can also include a post-translational modification. In one example, the post-translational modification is glycosylation and the CPG15-2 protein can be glycosylated at one, two, three, four, five, six or more amino acid sites in the protein. For the mouse protein, particularly preferred glycosylation sites include the alanine residue at amino acid 30 and the arginine residue at amino acid 68. In another example, the post-translational modification includes attachment of any membrane component such as lipids, proteins, phospholipids, or phosphoproteins, or any fragment thereof. In another embodiment, the CPG 15-2 protein can be a monomer, a homodimer, or a heterodimer with a different protein (e.g., CPG15 or s-CPG15).

In additional aspects, the invention features a composition that includes any of the CPG15-2 proteins described above and a pharmaceutically acceptable carrier.

The invention also features a method of manufacturing CPG 15-2 protein that includes expressing the CPG 15-2 protein in a population of cells and isolating the CPG15-2 from the cell population. In preferred embodiments, the CPG15-2 is at least 80%, preferably 85%, 90%, 95%, 99% or more pure. Desirably, the cells are neuronal cells or hippocampal cells; COS cells; CV-1 cells; L cells; C127 cells; 3T3 cells; CHO cells; HeLa cells; 293 cells; 293T cells; and BHK cells. The CPG15-2 manufactured by this method CPG15-2 can be any form of the CPG15-2 protein described above.

In another aspect, the invention provides an isolated nucleic acid molecule encoding a CPG15-2 protein that includes a sequence that is substantially identical (e.g., 85%, 90%, 95%, 99% or more) to the amino acid sequence of SEQ ID NOs: 2 or 4. Desirably, the isolated nucleic acid includes a sequence that encodes a protein having the sequence set forth in SEQ ID NOs: 2 or 4. In one embodiment, the isolated nucleic acid molecule can encode a protein that includes a sequence that is substantially identical (e.g., 85%, 90%, 95%, 99% or more) to the amino acid sequence of SEQ ID NOs: 5 or 6. The isolated nucleic acid molecule can include a sequence encoding an amino acid substantially identical (e.g., 85%, 90%, 95%, 99% or more) to amino acids 35 to 131 of mouse CPG15-2 (SEQ ID NO: 4) or a sequence encoding an amino acid substantially identical (e.g., 85%, 90%, 95%, 99% or more) to amino acids 38 to 134 of human CPG15-2 (SEQ ID NO: 2). The nucleic acid molecule can include a nucleic acid sequence that is substantially identical (e.g., 85%, 90%, 95%, 99%, or 100%) to the DNA sequence of human cpg15-2 (SEQ ID NO: 1) or mouse cpg15-2 (SEQ ID NO: 3). The isolated nucleic acid molecule can also be one that hybridizes under high stringency to a nucleic acid comprising the nucleotide sequence of SEQ ID NOs: 1 or 3.

In additional related embodiments, the invention provides a vector, a cell, a cell including the vector, a cell including any of the nucleic acids described above, or a non-human transgenic animal including the isolated nucleic acid sequences described above.

The invention also features a pharmaceutical composition comprising at least one dose of a therapeutically effective amount of CPG15-2 protein, or a fragment thereof, or a nucleic acid molecule encoding a CPG15-2 protein, or a fragment thereof, in a pharmaceutically acceptable carrier.

The invention also features a kit that includes a substantially pure CPG15-2 protein or a fragment thereof, and directions for the use of the protein for the treatment or prevention of a condition of excessive cell death. The CPG 15-2 protein can be any of the proteins or forms of the protein described above.

The invention also features a kit that includes an isolated nucleic acid molecule encoding a protein or fragment thereof with CPG 15-2 biological activity and directions for the use of the nucleic acid molecule for the treatment or prevention of a condition of excessive cell death. In preferred embodiments, the kit contains an isolated nucleic acid molecule having the sequence set forth in SEQ ID NO: 1 or 3.

Any of the compositions of the invention are preferably formulated with a pharmaceutically acceptable excipient.

The invention also generally features methods using the above CPG15-2 proteins and compositions for treating or preventing a condition of excessive cell death in a subject and for reducing or preventing cell death in general.

In one such aspect, the invention features a method of treating or preventing a condition of excessive cell death in a subject that includes administering to a subject CPG 15-2 in an amount and for a time sufficient to prevent, reduce, or eliminate the symptoms of the condition. In preferred embodiments, the cell death is mediated by apoptosis and can be measured by any standard apoptotic assay such as those described herein. A reduction in cell death typically includes at least a 5% decrease, preferably at least a 10%, 20%, 40%, 50%, 60%, 80%, or 100% decrease in the amount of cell death as compared to a control.

In additional preferred embodiments, the condition is a neurological condition including, but not limited to, any of the following: Alzheimer's Disease, Parkinson's Disease, Huntington's Disease, Amyotrophic Lateral Sclerosis, a condition of the retina and optic nerve, such as retinitis pigmentosa or macular degeneration; traumatic injury to the brain, and stroke. The condition can also be a condition of the bone, skin, muscle, joint, or cartilage; a cardiac condition, such as cardiac ischemia; an autoimmune condition; a liver condition; aging or an aging related condition; a condition characterized by ischemia; or an immunodeficiency condition.

Preferred dosages of CPG15-2 include 0.01 μg/kg to about 50 mg/kg per day, preferably 0.01 mg/kg to about 30 mg/kg per day, most preferably 0.1 mg/kg to about 20 mg/kg per day. The CPG15-2 may be given daily (e.g., once, twice, three times, four times daily, or continuously) or less frequently (e.g., once every other day, once or twice weekly, or monthly).

In another such aspect, the invention features a method of reducing or preventing cell death that includes administering to a cell CPG 15-2 in an amount and for a time sufficient to reduce or prevent cell death. In preferred embodiments, the cell death is mediated by apoptosis and can be measured by any standard apoptotic assay such as those described herein. A reduction in cell death typically includes at least a 5% decrease, preferably at least a 10%, 20%, 40%, 50%, 60%, 80%, or 100% decrease in the amount of cell death as compared to a control not treated with CPG 15-2.

In yet another such aspect, the invention features a method of promoting the survival or differentiation of a cell comprising administering to the cell CPG15-2 for a time and in an amount sufficient to promote the survival or differentiation of the cell. In preferred embodiments the cell is a tissue culture cell and the CPG15-2 is added to the culture media. An increase in the survival or differentiation of the cell is considered at least a 5%, preferably at least 10%, 20%, 40%, 50%, 60%, 80%, or 100% increase in the number of cells surviving or induced to differentiate as compared to a control population as measured using standard assays such as those described herein.

In preferred embodiments of the above two aspects, the cell is selected from the group consisting of a cell of the nervous system (e.g., a neuron such as a central nervous system neuron, a peripheral nervous system neuron, or a spinal cord neuron), muscle cell, stem cell, immune cell, blood cell, endothelial cell, fibroblast cell, epithelial cell, bone cell, skin cell, pancreatic cell, liver cell, cardiomyocyte, oligodendrocyte, and chondrocyte.

In preferred embodiments of any of the methods above, the CPG15-2 protein can include any of the forms of CPG15-2 described above.

The invention also features a method of treating or preventing a condition of excessive cell death in a subject that includes administering to the subject a nucleic acid molecule encoding a protein having CPG15-2 biological activity in an amount and for a time to prevent, reduce, or eliminate the symptoms of the condition. Preferably, the protein having CPG15-2 biological activity is encoded by a nucleic acid molecule operably linked to a promoter in a recombinant vector. The recombinant vector can be a viral vector derived from a virus such as adenovirus, adeno-associated virus, and lentivirus. Desirably, the nucleic acid can be any of the isolated nucleic acids described above.

The invention also features a purified antibody or antigen-binding fragment thereof that specifically binds CPG15-2. The antibody or antigen-binding fragment thereof can be a monoclonal antibody or a polyclonal antibody. Monoclonal and polyclonal antibodies may be produced by methods known in the art. These methods include the immunological method described by Kohler and Milstein (Nature, 256: 495-497, 1975) and Campbell (“Monoclonal Antibody Technology, The Production and Characterization of Rodent and Human Hybridomas” in Burdon et al., Eds., Laboratory Techniques in Biochemistry and Molecular Biology, Volume 13, Elsevier Science Publishers, Amsterdam, 1985), as well as by the recombinant DNA method described by Huse et al. (Science, 246, 1275-1281, 1989).

Monoclonal antibodies may be prepared from supernatants of cultured hybridoma cells or from ascites induced by intraperitoneal inoculation of hybridoma cells into mice. The hybridoma technique described originally by Kohler and Milstein (Eur. J. Immunol, 6, 511-519, 1976) has been widely applied to produce hybrid cell lines that secrete high levels of monoclonal antibodies against many specific antigens.

In another aspect, the invention features a purified nucleic acid molecule having at least one strand that is at least 80%, preferably 85%, 90%, 95%, 99%, or 100% complementary to at least a portion of any of one of the sequences set forth in SEQ ID NOs: 1 and 3, and where the nucleic acid molecule can reduce to reduce or inhibit the expression or biological activity of a CPG 15-2 protein in a cell. Desirably, the nucleic acid molecule has at least one strand that is at least 80%, preferably 85%, 90%, 95%, 99%, or 100% complementary to nucleotides 1 to 72 or 357 to 608 of SEQ ID NO: 3 or nucleotides 1 to 132 or 361 to 608 of SEQ ID NO: 1. In additional embodiments, where a nucleic acid that can reduce or inhibit the expression or biological activity of both CPG15-2 and CPG15 is desired, the nucleic acid can have at least one strand that is complementary to nucleotides 73 to 356 of SEQ ID NO: 3 or nucleotides 133 to 360 of SEQ ID NO: 1. In preferred embodiments, the nucleic acid molecule is a double stranded RNA molecule (dsRNA), more preferably an siRNA molecule. Desirably, the siRNA molecule is 100% complementary to at least 18, preferably 19, 20, 21, 22, 23, 24, 25, 35, 45, 50 or more consecutive nucleotides of any one the sequence set forth in SEQ ID NOs: 1 and 3. In a preferred embodiment, the siRNA has at least one strand that is 100% complementary to 18 to 25 consecutive nucleotides of SEQ ID NOs: 1 or 3. The dsRNA molecule can also be a short hairpin RNA (shRNA).

In another embodiment, the nucleic acid molecule is an antisense nucleobase oligomer molecule. Desirably, the antisense nucleobase oligomer is 80%, 85%, 90%, 95%, or 100% complementary to at least 10, preferably 20, 30, 40, 50, 60, 70, 80, 90, 100, 200, 300, or more consecutive nucleotides of any one the sequence set forth in SEQ ID NOs: 1 and 3. Preferred antisense nucleobase oligomers are 8 to 30 nucleotides in length.

In preferred embodiments of any of the above aspects, the antibody or antigen binding fragment or the nucleic acid is formulated with a pharmaceutically acceptable carrier.

The invention also features a kit that includes a purified antibody or antigen-binding fragment thereof that specifically binds CPG 15-2, as described above, and directions for its use for the treatment or prevention of a condition of undesirable cell survival.

The invention also features a kit that includes a purified nucleic acid molecule as described above and directions for its use for the treatment or prevention of a condition of undesirable cell survival.

The invention also generally features the use of the antibodies and nucleic acid molecules that can reduce to reduce or inhibit the expression or biological activity of a CPG 15-2 protein in a cell, as described above, for methods of treating or preventing a condition of undesirable cell survival in a subject.

In one such aspect, the invention features a method of treating or preventing a condition of undesirable cell survival in a subject that includes administering to the subject a purified antibody or antigen-binding fragment that specifically binds a polypeptide having CPG 15-2 biological activity for an amount and for a time sufficient to prevent, reduce, or eliminate the symptoms of the condition. In preferred embodiments, the condition is cancer, tumor-associated angiogenesis, or an immune system condition.

In another such aspect, the invention features a method of treating or preventing a condition of undesirable cell survival in a subject that includes administering to the subject a nucleic acid molecule having at least one strand that is at least 80%, preferably 85%, 90%, 95%, 99%, or 100% complementary to at least a portion of any of one of the sequences set forth in SEQ ID NOs: 1 and 3, in an amount and for a time sufficient to reduce or inhibit the expression of a CPG 15-2 protein in a cell. In one preferred embodiments, the nucleic acid molecule is an antisense nucleobase oligomer molecule as described above. Desirably, the antisense nucleobase oligomer has at least one strand that is 100% complementary to 8 to 30 consecutive nucleotides of any one the sequence set forth in SEQ ID NOs: 1 and 3. In preferred embodiments, the condition is cancer, tumor-associated angiogenesis, or an immune system condition.

In another aspect, the nucleic acid molecule used for the treatment or prevention of a condition of undesirable cell survival in a subject is a double stranded RNA molecule, as described above, that is administered in an amount and for a time sufficient to reduce or inhibit the expression or biological activity of a CPG 15-2 protein in a cell. Desirably, the ds RNA is provided as or processed into small interfering RNAs 18 to 25 nucleotides in length or is a short hairpin RNA. In a preferred embodiment, the siRNA has at least one strand that is 100% complementary to 18 to 25 consecutive nucleotides of SEQ ID NOs: 1 or 3. In preferred embodiments, the condition is cancer, tumor-associated angiogenesis, or an immune system condition.

In yet another such aspect, the invention features a method of treating or preventing a condition of undesirable cell survival in a subject, comprising administering to the subject a truncated form of CPG15-2 or a nucleic acid encoding a truncated form of CPG15-2 in an amount and for a time sufficient to reduce or inhibit the biological activity of CPG 15-2. Preferred truncated forms will reduce or inhibit the biological activity of CPG15-2 by at least 10%, 20%, 30%, 40%, 50%, 60%, 70%, 80%, 90% or more, as measured by any of the assays described herein or known in the art. In preferred embodiments, the condition is cancer, tumor-associated angiogenesis, or an immune system condition. Preferred truncated forms include an amino acid sequence that has at least 50, 60, or 75%, more preferably at least 80, 85, or 95%, and most preferably at least 99% amino acid identity to any of the following sequences.

Mouse CPG15-2 with signal peptide, without GPI anchor:

(SEQ ID NO: 19)

MMCNCCHCHWRRRCQRLPCALTLLLLLPLAVASEGPNRCDTIYQGFAECL
IRLGDGMGRGGELQTVCRSWNDFHACASRVLSGCPEEAAAVWESLQQEAR
RAPHPDNLHILCGAPVSVRERIAGPETNQETLRATA;

mouse CPG15-2 without signal peptide, without GPI anchor:

(SEQ ID NO: 5)

SEGPNRCDTIYQGFAECLIRLGDGMGRGGELQTVCRSWNDFHACASRVLS
GCPEEAAAVWESLQQEARRAPHPDNLHILCGAPVSVRERIAGPETNQETL
RATA;

human CPG15-2 with signal peptide, without GPI anchor:

(SEQ ID NO: 20)

MMRCCRRRCCCRQPPHALRPLLLLPLVLLPPLAAAAAGPNRCDTIYQGFA
ECLIRLGDSMGRGGELETICRSWNDFHACASQVLSGCPEEAAAVWESLQQ
EARQAPRPNNLHTLCGAPVHVRERGTGSETNQETLRATA;
and

human CPG 15-2 without signal peptide, without GPI anchor:

(SEQ ID NO: 6)

AAGPNRCDTIYQGFAECLIRLGDSMGRGGELETICRSWNDFHACASQVLS
GCPEEAAAVWESLQQEARQAPRPNNLHTLCGAPVHVRERGTGSETNQETL
RATA.

In preferred embodiments of any of the above aspects, a reduction in undesirable cell survival includes at least a 5% decrease, preferably at least a 10%,20%, 40%, 50%, 60%, 80%, or 100% decrease in the number of cells as compared to a control when measured by standard art-known cell survival or cell death assays or the assays described herein.

In preferred aspects of any of the methods of the invention, a subject includes humans and other animals, preferably warm-blooded mammals including mice, rats, guinea pigs, hamsters, rabbits, cats, dogs, goats, sheep, cows, or monkeys.

In additional aspects, the invention features methods of enhancing cell death comprising administering to a cell any one of the following: an antibody or antigen-binding fragment that specifically binds CPG15-2; an antisense nucleobase oligomer complementary to a nucleic acid sequence encoding a protein having CPG 15-2 biological activity; a double stranded RNA that is complementary to an mRNA sequence encoding a protein having CPG15-2 biological activity; a nucleic acid molecule encoding a truncated form of CPG15-2; or a truncated form of CPG15-2. Any or all of the above are administered in an amount sufficient to reduce or inhibit the biological activity of CPG 15-2 or to enhance cell death. The above aspects can be used to treat any condition characterized by undesirable cell survival (e.g., cancer, tumor-associated angiogenesis, or an immune system condition). In any of the above aspects, cell death is measured by standard apoptotic assays such as those described herein and a increase in cell death is at least a 5% increase, preferably at least a 10%, 20%, 40%, 50%, 60%, 80%, or 100% increase in the number of cells undergoing apoptosis.

In addition, the invention features a method of identifying candidate compounds that regulate cell death, cell survival, or cellular differentiation pathways. The method includes the steps of (a) mixing CPG 15-2 with a test mixture and (b) identifying a candidate compound in the test mixture that interacts with the CPG15-2. In preferred embodiments, the method also includes the step after step (a) of incubating the CPG15-2/test mixture with an insoluble affinity support reagent that specifically binds CPG 15-2. In additional preferred embodiments the method also includes the step of recovering the affinity support reagent bound to CPG 15-2. The test mixture can be a cell lysate or a lysate from a tissue. The modulation of cell death, cell survival, or cell differentiation can be an increase or a decrease as measured by standard apoptosis, cell survival, or cell differentiation assays such as those described herein. Preferably the increase or decrease will result in a change of at least 5%, more preferably at least 10%, 20%, 40%, 50%, 60%, 80%, or 100%.

By “antisense nucleobase oligomer” is meant a nucleobase oligomer, regardless of length, that is complementary to the coding strand or mRNA of a gene that encodes a protein having CPG15-2 biological activity. By a “nucleobase oligomer” is meant a compound that includes a chain of at least eight nucleobases, preferably at least twelve, and most preferably at least sixteen bases, joined together by linkage groups. Included in this definition are natural and non-natural oligonucleotides, both modified (e.g., phosphorothioates, phosphorodithioates, and phosphotriesters) and unmodified, oligonucleotides with modified (e.g., morpholino linkages and heteroatom backbones) or unmodified backbones, as well as oligonucleotide mimetics such as Protein Nucleic Acids, locked nucleic acids, and arabinonucleic acids. Numerous nucleobases and linkage groups may be employed in the nucleobase oligomers of the invention, including those described in U.S. Patent Publication Nos. 20030114412 (see for example paragraphs 27-45 of the publication) and 20030114407 (see for example paragraphs 35-52 of the publication), incorporated herein by reference. The nucleobase oligomer can also be targeted to the translational start and stop sites. Preferably the antisense nucleobase oligomer comprises from about 8 to 30 nucleotides. The antisense nucleobase oligomers can also contain at least 40, 60, 85, 120, or more consecutive nucleotides that are complementary to the mRNA or DNA that encodes a protein having CPG15-2 biological activity, and may be as long as the full-length mRNA or gene.

By “apoptosis” or “apoptotic cell death” is meant the process of cell death wherein a dying cell displays a set of well-characterized biochemical hallmarks that include cell membrane blebbing, cell soma shrinkage, chromatin condensation, and DNA laddering. Cells that die by apoptosis include neurons (e.g., during the course of neurodegenerative diseases or neurogenesis), cardiomyocytes (e.g., after myocardial infarction or over the course of congestive heart failure), immune cells (e.g., after HIV infection), and cancer cells (e.g., after exposure to radiation or chemotherapeutic agents).

By “candidate plasticity gene 15-2” or “cpg15-2” is meant a nucleic acid molecule, such as a genomic DNA, cDNA, or RNA (e.g., mRNA) molecule, having at least 50, 60, or 75%, more preferably at least 80, 85, or 95%, and most preferably at least 99% nucleic acid identity to either of the nucleic acid molecules set forth in SEQ ID NOs: 1 and 3 or to a nucleic acid molecule encoding the proteins set forth in SEQ ID NOs: 2 or 4.

As used herein, the term “nucleic acid molecule” refers to any nucleic acid containing molecule including, but not limited to DNA or RNA. The term encompasses sequences that include any of the known base analogs of DNA and RNA including, but not limited to, 4-acetylcytosine, 8-hydroxy-N-6-methyladenosine, aziridinylcytosine, pseudoisocytosine, 5-(carboxyhydroxylmethyl) uracil, 5-fluorouracil, 5-bromouracil, 5-carboxymethylaminomethyl-2-thiouracil, 5-carboxymethylaminomethyluracil-, dihydrouracil, inosine, N6-isopentenyladenine, 1-methyladenine, 1-methylpseudouracil, 1-methylguanine, 1-methylinosine, 2,2-dimethylguanine, 2-methyladenine, 2-methylguanine, 3-methylcytosine, 5-methylcytosine, N6-methyladenine, 7-methylguanine, 5-methylaminomethyluracil, 5-methoxyaminomethyl-2-thiouracil, beta-D-mannosylqueosine, 5′-methoxycarbonylmethyluracil, 5-methoxyuracil, 2-methylthio-N6-isopentenyladenine, uracil-5-oxyacetic acid methylester, uracil-5-oxyacetic acid, oxybutoxosine, pseudouracil, queosine, 2-thiocytosine, 5-methyl-2-thiouracil, 2-thiouracil, 4-thiouracil, 5-methyluracil, N-uracil-5-oxyacetic acid methylester, uracil-5-oxyacetic acid, pseudouracil, queosine, 2-thiocytosine, and 2,6-diaminopurine. The term nucleic acids also includes any modification that enhances the stability or function of the nucleic acid in any way. Examples include modifications to the phosphate backbone, the internucleotide linkage, or to the sugar moiety.

By “CPG15-2” is meant a protein that is encoded by cpg15-2, or a substantially identical nucleic acid molecule, and that has at least 50, 60, or 75%, more preferably at least 80, 85, or 95%, and most preferably at least 99% amino acid identity to either of the proteins set forth in SEQ ID NOs: 2 and 4 or GenBank accession numbers NM_{—}198443 (human) or AK090312 (mouse) and that has CPG 15-2 biological activity. Naturally occurring variants are also embodied in the methods and compositions of the invention, particularly those having CPG15-2 biological activity. CPG15-2 includes all forms that possess CPG15-2 biological activity such as the membrane bound CPG15-2, soluble CPG15-2, and modified CPG15-2. The membrane bound form of CPG15-2 is a naturally secreted, processed form of the protein that lacks the signal sequence and that has a cleaved GPI sequence but is anchored to the plasma membrane via a lipid moiety. The soluble form, s-CPG15-2, is a naturally secreted form that is soluble, and has the ability to promote cell survival or protect a cell from cell death. s-CPG 15-2 generally refers to the core domain of the protein after cleavage of the GPI linkage sequences or the secretion signal sequence, or both. These sequences are typically cleaved off after translation and processing of the protein. The remaining sequences after cleavage are known as the core domain. For mouse CPG 15-2 the core domain can include an amino acid sequence that has at least 50, 60, or 75%, more preferably at least 80, 85, or 95%, and most preferably at least 99% amino acid identity to the following sequence: SEGPNRCDTIYQGFAECLIRLGDGMGRGGELQTVCRSWNDFHACASRVLSGCPE EAAAVWESLQQEARRAPHPDNLHILCGAPVSVRERIAGPETNQETLRATA (SEQ ID NO: 5). For human CPG15-2 the core domain can include an amino acid sequence that has at least 50, 60, or 75%, more preferably at least 80, 85, or 95%, and most preferably at least 99% amino acid identity to the following sequence:

(SEQ ID NO: 6)

AAGPNRCDTIYQGFAECLIRLGDSMGRGGELETICRSWNDFHACASQVLS
GCPEEAAAVWESLQQEARQAPRPNNLHTLCGAPVHVRERGTGSETNQETL
RATA.

In general, the soluble form of CPG15-2 is produced via cleavage of the membrane bound form of CPG15-2 causing release of the protein from the membrane into the extracellular space. s-CPG 15-2 is typically purified from the supernatant of growing cells.

In preferred embodiments, CPG 15-2 includes any modifications to the protein (e.g., the carboxy-terminus of the protein) that occur before or after localization to the plasma membrane. Such modifications can include, for example, post-translational modifications to the protein including but not limited to phosphorylation, hydroxylation, sulfation, acetylation, glycosylation, subunit dimerization (homodimers or heterodimers with additional proteins such as CPG 15 or s-CPG 15) or multimerization, and cross-linking or attachment to any other proteins, or fragments thereof, or membrane components, or fragments thereof (e.g., cleavage of the protein from the membrane with a lipid component attached). By “truncated CPG15-2 (t-CPG15-2)” is meant any non-natural form of CPG 15-2 that lacks the amino acids encoding the GPI linkage sequence (e.g., the last 26 amino acids of mouse CPG15-2, see FIG. 1C). In general, t-CPG15-2 is expressed from an engineered construct containing the nucleic acid sequence encoding CPG 15-2 but lacking the nucleotides that encode the GPI linkage sequence. This truncated form of CPG 15-2 does not follow the GPI linkage pathway but is instead secreted directly out of the cell without membrane attachment or modifications associated with membrane attachment. t-CPG 15-2 can function as a dominant negative to inhibit CPG15-2 biological activity using the assays described herein. Exemplary forms of truncated CPG15-2 include the following:

Truncated CPG15-2 can include an amino acid sequence that has at least 50, 60, or 75%, more preferably at least 80, 85, or 95%, and most preferably at least 99% amino acid identity to the following mouse CPG 15-2 sequences:

(SEQ ID NO: 5)

SEGPNRCDTIYQGFAECLIRLGDGMGRGGELQTVCRSWNDFHACASRVLS
GCPEEAAAVWESLQQEARRAPHPDNLHILCGAPVSVRERIAGPETNQETL
RATA
or

(SEQ ID NO: 19)

MMCNCCHCHWRRRCQRLPCALTLLLLLPLAVASEGPNRCDTIYQGFAECL
IRLGDGMGRGGELQTVCRSWNDFHACASRVLSGCPEEAAAVWESLQQEAR
RAPHPDNLHILCGAPVSVRERIAGPETNQETLRATA.

### Truncated CPG15-2 can also include an amino acid sequence that has at least 50, 60, or 75%, more preferably at least 80, 85, or 95%, and most preferably at least 99% amino acid identity to the following human CPG15 sequences:

(SEQ ID NO: 6)

AAGPNRCDTIYQGFAECLIRLGDSMGRGGELETICRSWNDFHACASQVLS
GCPEEAAAVWESLQQEARQAPRPNNLHTLCGAPVHVRERGTGSETNQETL
RATA
or

(SEQ ID NO: 20)

MMRCCRRRCCCRQPPHALRPLLLLPLVLLPPLAAAAAGPNRCDTIYQGFA
ECLIRLGDSMGRGGELETICRSWNDFHACASQVLSGCPEEAAAVWESLQQ
EARQAPRPNNLHTLCGAPVHVRERGTGSETNQETLRATA.

By “CPG15-2 biological activity” is meant the ability to promote cell survival or to prevent or reduce cell death. The biological activity of CPG 15-2, or fragments thereof, can be assayed using standard cell death assays such as the apoptosis, necrosis, and cell starvation assays as described herein. In addition, CPG15-2 biological activity can further include the ability to promote growth and differentiation. These functions can also be measured using standard art known assays such as those described herein. One exemplary assay to measure neuronal cell growth and differentiation is the in vitro explant assay for process outgrowth as described in Placzek et al., (Science 250:985-988, 1990); Ringstedt et al., (J. Neurosci. 20:4983-4991, 2000); Charron et al., (Cell 113:11-23, 2003); and Wang et al., (Nature 401:765-769, 1999). Tissue sources used in these assays can also be obtained using the methods described in Baranes et al., (Proc. Natl. Acad. Sci. 93:4706-4711, 1996). “In vitro CPG 15-2 biological activity” can also be measured using the cell starvation assays described herein, the in vitro explant assay for process outgrowth, or the neurite outgrowth and survival assays as described herein. A protein having CPG 15-2 biological activity will preferably rescue at least 10% of the cells from cell death, more preferably at least 20%, 30%, 50%, 75% or more. For the cell growth and survival assyas, a protein having CPG 15-2 biological activity will preferably induce survival in at least 10% of the cells, more preferably at least 20%, 30%, 50%, 75% or more.

By “cell death” is meant the process or series of events, which ultimately lead to a non-functioning, non-living cell. Cell death as used herein typically refers to apoptosis (programmed cell death) or necrosis. By “preventing or reducing” cell death is meant any treatment or therapy that causes an overall decrease in the number of cells undergoing cell death relative to a control. Preferably, the decrease will be at least 15%, more preferably at least 25%, and most preferably at least 50%. By “excessive cell death” is meant an increase in the number of cells undergoing cell death as compared to a control population of cells. Preferably, excessive cell death includes an increase of 10% or more in the total number of cells undergoing cell death. More preferably the increase is 15%, 20%, 25% or more, and most preferably an increase of 40% or more in the total number of cells undergoing cell death as compared to a control population of cells. Cell death can be measured by any of the standard assays known in the art such as those described herein.

By “cell survival” is meant the reversal or prevention of cell death signaling pathways or the promotion of pathways that antagonize cell death, thereby increasing the life span of a cell or the number of cells that survive in a given situation, relative to a control. By “cell survival” is also meant the induction of cell growth or cell proliferation pathways. By “promoting” cell survival is meant any treatment or therapy that causes an overall increase in the number of cells. Preferably, the increase will be at least 15%, more preferably at least 25%, and most preferably at least 50%. “Undesirable cell survival” is characterized by an increase in cell proliferation or a decrease in cell death such that the total number of growing cells exceeds that of a normal control population. Preferably, “undesirable cell survival” refers to an increase of 15% or more in the total number of growing cells. More preferably the increase is 25% or more and most preferably the total number of growing cells will be 50% or more than the number of growing cells in a control population. Preferably, changes in cell survival and cell death are measured using standard cell survival assays or apoptosis assays such as the serum starvation assay or the trypan blue staining assay described herein below.

By “differentiation” is meant the process during which young, immature (unspecialized) cells take on individual characteristics and reach their mature (specialized) form and function. By “promoting” cell differentiation is meant any treatment or therapy that causes an overall increase in the number of differentiated cells as measured by assays which quantitate the presence or absence of a defining characteristic of a differentiated cell. Preferably, the increase in differentiation of a cell population will be at least 15%, more preferably at least 25%, and most preferably at least 50%. In one example, stem cell conversion to neurons can be measured by expression of neuronal markers such as neurofilament-M, Map2, and neuron specific enolase. In another example, the clonogenic Colony Assay offered by Cambrex Corporation, can be used to determine differentiation of hematopoietic progenitor cells into myeloid (CFU-GM), erythroid (CFU-E, BFU-E), megakaryocyte (CFU-Meg), and mixed (myeloid and erythroid) colonies.

By “complementary” or “complementarity” is meant polynucleotides (i.e., a sequence of nucleotides) related by the nucleobase-pairing rules. For example, for the sequence “A-G-T,” is complementary to the sequence “T-C-A.” Complementarity may be “partial,” in which only some of the nucleic acid bases are matched according to the base pairing rules. In preferred embodiments, a partially or substantially complementary nucleic acid molecule has at least 80%, preferably 85%, 90%, 95%, or 99% of its bases matched to the bases in the comparison molecule according to the base pairing rules. Or, there may be “complete” or “total” complementarity between the nucleic acids. Sequence complementarity is measured using the same methods as described for measuring sequence identity, below. In a preferred embodiment, sequence complementarity is measured for a given number of consecutive residues and excludes additional residues such as overhang residues.

By “hybridize” is meant pair to form a double-stranded molecule between complementary polynucleotide sequences, or portions thereof, under various conditions of stringency. (See, e.g., Wahl, G. M. and S. L. Berger (1987) Methods Enzymol. 152:399; Kimmel, A. R. (1987) Methods Enzymol. 152:507) For example, stringent salt concentration will ordinarily be less than about 750 mM NaCl and 75 mM trisodium citrate, preferably less than about 500 mM NaCl and 50 mM trisodium citrate, and most preferably less than about 250 mM NaCl and 25 mM trisodium citrate. Low stringency hybridization can be obtained in the absence of organic solvent, e.g., formamide, while high stringency hybridization can be obtained in the presence of at least about 35% formamide, and most preferably at least about 50% formamide. Stringent temperature conditions will ordinarily include temperatures of at least about 30° C., more preferably of at least about 37° C., and most preferably of at least about 42° C. Varying additional parameters, such as hybridization time, the concentration of detergent, e.g., sodium dodecyl sulfate (SDS), and the inclusion or exclusion of carrier DNA, are well known to those skilled in the art. Various levels of stringency are accomplished by combining these various conditions as needed. In a preferred embodiment, hybridization will occur at 30° C. in 750 mM NaCl, 75 mM trisodium citrate, and 1% SDS. In a more preferred embodiment, hybridization will occur at 37° C. in 500 mM NaCl, 50 mM trisodium citrate, 1% SDS, 35% formamide, and 100 μg/ml denatured salmon sperm DNA (ssDNA). In a most preferred embodiment, hybridization will occur at 42° C. in 250 mM NaCl, 25 mM trisodium citrate, 1% SDS, 50% formamide, and 200 μg/ml ssDNA. Useful variations on these conditions will be readily apparent to those skilled in the art.

For most applications, washing steps that follow hybridization will also vary in stringency. Wash stringency conditions can be defined by salt concentration and by temperature. As above, wash stringency can be increased by decreasing salt concentration or by increasing temperature. For example, stringent salt concentration for the wash steps will preferably be less than about 30 mM NaCl and 3 mM trisodium citrate, and most preferably less than about 15 mM NaCl and 1.5 mM trisodium citrate. Stringent temperature conditions for the wash steps will ordinarily include a temperature of at least about 25° C., more preferably of at least about 42° C., and most preferably of at least about 68° C. In a preferred embodiment, wash steps will occur at 25° C. in 30 mM NaCl, 3 mM trisodium citrate, and 0.1% SDS. In a more preferred embodiment, wash steps will occur at 42° C. in 15 mM NaCl, 1.5 mM trisodium citrate, and 0.1% SDS. In a most preferred embodiment, wash steps will occur at 68° C. in 15 mM NaCl, 1.5 mM trisodium citrate, and 0.1% SDS. Additional variations on these conditions will be readily apparent to those skilled in the art. Hybridization techniques are well known to those skilled in the art and are described, for example, in Benton and Davis (Science 196:180, 1977); Grunstein and Hogness (Proc. Natl. Acad. Sci., USA 72:3961, 1975); Ausubel et al. (Current Protocols in Molecular Biology, Wiley Interscience, New York, 2001); Berger and Kimmel (Guide to Molecular Cloning Techniques, 1987, Academic Press, New York); and Sambrook et al., Molecular Cloning: A Laboratory Manual, Cold Spring Harbor Laboratory Press, New York.

By “membrane component” is meant any lipid (e.g., cholesterol), glycolipid, protein, phospholipid, or phosphoprotein or any fraction thereof that is found in a cell membrane.

By “necrosis” or “necrotic cell death” is meant cell death associated with a passive process involving loss of integrity of the plasma membrane and subsequent swelling, followed by lysis of the cell.

By “neurological condition” is meant any condition of the central or peripheral nervous system that is associated with neuron degeneration or damage. Specific examples of neurological conditions include, but are not limited to, Alzheimer's disease, Parkinson's disease, Huntington's disease, ALS, peripheral neuropathies, stroke, trauma, and other conditions characterized by neuronal death or loss of neurons, whether central peripheral, or motor neurons. Neuronal conditions also include conditions of the retina and optic nerve such as macular degeneration, retinal degeneration, retinitis pigmentosa, and general macular dystrophies.

By “operably linked” is meant that a gene and a regulatory sequence(s) are connected in such a way as to permit gene expression when the appropriate molecules (e.g., transcriptional activator proteins) are bound to the regulatory sequence(s).

By “pharmaceutically acceptable carrier” is meant a carrier that is physiologically acceptable to the treated mammal while retaining the therapeutic properties of the compound with which it is administered. One exemplary pharmaceutically acceptable carrier substance is physiological saline. Other physiologically acceptable carriers and their formulations are known to one skilled in the art and described, for example, in Remington's Pharmaceutical Sciences, (20^{th} edition), ed. A. Gennaro, 2000, Lippincott, Williams & Wilkins, Philadelphia, Pa.

By “reduce” or “inhibit” is meant the ability to cause an overall decrease by 20%, 30%, or 40%, more preferably by 50%, 60%, or 70%, most preferably by 80%, 90%, or even 100% in the level of protein or nucleic acid as compared to samples not treated with the nucleic acid molecules of the invention. This reduction or inhibition of RNA or protein expression can occur through targeted mRNA cleavage or degradation. Assays for protein expression or nucleic acid expression are known in the art and include, for example, ELISA and western blot analysis for protein expression, Southern blotting or PCR for DNA analysis, and northern blotting, PCR, or RNase protection assays for RNA.

By “small interfering RNAs (siRNAs)” is meant an isolated dsRNA molecule, preferably greater than 10 nucleotides in length, more preferably greater than 15 nucleotides in length, and most preferably greater than 19 nucleotides in length, that is used to identify the target gene or mRNA to be degraded. A range of 19-25 nucleotides is the most preferred size for siRNAs. siRNAs can also include short hairpin RNAs in which both strands of an siRNA duplex are included within a single RNA molecule via a base linker region. siRNA includes any form of dsRNA (proteolytically cleaved products of larger dsRNA, partially purified RNA, essentially pure RNA, synthetic RNA, recombinantly produced RNA) as well as altered RNA that differs from naturally occurring RNA by the addition, deletion, substitution, and/or alteration of one or more nucleotides. Such alterations can include the addition of non-nucleotide material, such as to the end(s) of the RNA molecule or internally (at one or more nucleotides of the RNA). In a preferred embodiment, the RNA molecules contain a 3′hydroxyl group. Nucleotides in the RNA molecules of the present invention can also comprise non-standard nucleotides, including non-naturally occurring nucleotides or deoxyribonucleotides. The double-stranded oligonucleotide may contain a modified backbone, for example, phosphorothioate, phosphorodithioate, or other modified backbones known in the art, or may contain non-natural internucleoside linkages. Additional modifications of siRNAs (e.g., 2′-O-methyl ribonucleotides, 2′-deoxy-2′-fluoro ribonucleotides, “universal base” nucleotides, 5-C-methyl nucleotides, one or more phosphorothioate internucleotide linkages, and inverted deoxyabasic residue incorporation) can be found in the published U.S. Patent Publication Number 20040019001 (see Summary of the Invention section). Collectively, all such altered RNAs are referred to as modified siRNAs. In particular embodiments, siRNAs can be synthesized or generated by processing longer double-stranded RNAs, for example, in the presence of the enzyme dicer under conditions in which the dsRNA is processed to RNA molecules of about 18 to about 25 nucleotides. Collectively, all such altered RNAs are referred to as analogs of RNA. siRNAs of the present invention need only be sufficiently similar to natural RNA that it has the ability to mediate RNAi. As used herein “mediate RNAi” refers to the ability to distinguish or identify which RNAs are to be degraded.

Desirably, the antisense nucleobase oligomers or siRNA used for RNA interference will cause an overall decrease preferably of 20% or greater, more preferably of 50% or greater, and most preferably of 75% or greater, in the level of protein or nucleic acid, detected by standard art known assays, as compared to samples not treated with antisense nucleobase oligomers or dsRNA used for RNA interference. Examples of assays for protein expression include western blotting, examples of assays for RNA expression include northern blotting, PCR, and RNase protection assays, and examples of assays for DNA expression include Southern blotting and PCR.

By “specifically binds” is meant an antibody or antigen binding fragment thereof that recognizes and binds an antigen but that does not substantially recognize or bind to other molecules in a sample, e.g., a biological sample, that naturally includes protein. Specific recognition of an antigen by an antibody can be assayed using standard art known techniques such as immunoprecipitation, western blotting, and ELISA.

By “substantially identical” is meant a protein or nucleic acid exhibiting at least 50%, preferably 85%, more preferably 90%, and most preferably 95% sequence identity to a reference protein or nucleic acid sequence. For proteins, the length of comparison sequences will generally be at least 16 amino acids, preferably 20 amino acids, more preferably at least 25 amino acids and most preferably at least 35 amino acids. For nucleic acids the length of comparison sequences will generally be at least 10, 15, 20, 25, or 50 nucleotides, preferably at least 60 nucleotides, more preferably at least 75 nucleotides and most preferably 110 nucleotides or more. In preferred embodiments a nucleic acid or a protein that is substantially identical is substantially identical over its entire length.

Methods to determine identity are available in publicly available computer programs. Computer program methods to determine identity between two sequences include, but are not limited to, the GCG program package (Devereux et al., Nucleic Acids Research 12: 387, 1984), BLASTP, BLASTN, and FASTA (Altschul et al., J. Mol. Biol. 215:403 (1990). The well-known Smith Waterman algorithm may also be used to determine identity. The BLAST program is publicly available from NCBI and other sources (BLAST Manual, Altschul, et al., NCBI NLM NIH, Bethesda, Md. 20894; BLAST 2.0 at http://www.ncbi.nlm.nih.gov/blast/). These software programs match similar sequences by assigning degrees of homology to various substitutions, deletions, and other modifications. Conservative substitutions typically include substitutions within the following groups: glycine, alanine; valine, isoleucine, leucine; aspartic acid, glutamic acid, asparagine, glutamine; serine, threonine; lysine, arginine; and phenylalanine, tyrosine.

By “substantially pure” or “substantially pure and isolated” is meant a protein (or a fragment thereof) that has been separated from components that naturally accompany it. Typically, the protein is substantially pure when it is at least 60%, by weight, free from the proteins and naturally occurring organic molecules with which it is naturally associated. Preferably, the protein is at least 75%, more preferably at least 90%, and most preferably at least 99%, by weight, pure. Preferably, a substantially pure CPG15-2, will contain less than 20%, more preferably less than 10%, 5% or 1% of CPG15. A substantially pure CPG 15-2 protein may be obtained by standard techniques, for example, by extraction from a natural source (e.g., nervous system tissue or cell lines), by expression of a recombinant nucleic acid encoding a CPG 15-2 protein, or by chemically synthesizing the protein. Purity can be measured by any appropriate method, e.g., by column chromatography, polyacrylamide gel electrophoresis, or HPLC analysis.

By “therapeutic amount” is meant an amount that when administered to a patient suffering from the condition being treated is sufficient to cause a qualitative or quantitative reduction in the symptoms of the condition. A “therapeutic amount” can also mean an amount that when administered to a patient suffering from a condition of undesirable cell survival is sufficient to cause a reduction in the expression levels of CPG15-2 as measured by the assays described herein.

By “treating” is meant administering a compound or a pharmaceutical composition for prophylactic and/or therapeutic purposes. To “treat disease” or use for “therapeutic treatment” refers to administering treatment to a subject already suffering from a condition to improve the subject's condition. Preferably, the subject is diagnosed as suffering from a condition based on identification of any of the characteristic symptoms known for that condition and an effective treatment may prevent or reduce at least one symptom. In preferred embodiments, an effective treatment will reduce the symptom by at least 5%, preferably 25%, more preferably 50% or more. To “prevent disease” refers to prophylactic treatment of a subject who is not yet ill, but who is susceptible to, or otherwise at risk of, developing a particular condition. Thus, in the claims and embodiments, treating is the administration to a subject either for therapeutic or prophylactic purposes.

Other features and advantages of the invention will be apparent from the following description of the preferred embodiments thereof, and from the claims.

### BRIEF DESCRIPTION OF THE DRAWINGS

The application file contains at least one drawing executed in color (FIG. 3B). Copies of this patent or patent application with color drawings will be provided by the Office upon payment of the necessary fee.

FIG. 1A shows the DNA sequence (SEQ ID NO: 1) with the predicted amino acid translation of human cpg 15-2 (SEQ ID NO: 2). FIG. 1B shows the DNA sequence (SEQ ID NO: 3) with the predicted amino acid translation of mouse cpg 15-2 (SEQ ID NO: 4). FIG. 1C shows a comparison of the predicted mouse CPG15-2 and CPG15 amino acid sequences. Identical residues are marked by asterisks and similar residues are marked by dots. Conserved cysteines are boxed. Predicted signal peptides at the N-termini and GPI-anchoring signals at the C-termini are underlined. Predicted glycosylation sites on CPG15-2 are marked by arrowheads, and exon/intron boundaries are marked by arrows. FIG. 1D shows a comparison of mouse cpg15 and mouse cpg15-2 genomic structures. Exons, indicated by closed boxes, are numbered in sequence. FIG. 1E shows a phylogenetic analysis of the cpg15 gene family. All the family members in various species belong to either the cpg15 branch or the cpg15-2 branch. Of the species where the whole genome has been sequenced, human and mouse have two family members whereas the pufferfish has five family members. Accession numbers are indicated in parenthesis.

FIG. 2A is a Northern blot that shows the induction of cpg 15 and cpg15-2 mRNA in response to kainate injection. Poly (A) enriched RNA from cerebral cortices of uninjected (−) and kainate injected (KA) mice were analyzed for cpg15 and cpg15-2 expression by northern blotting. FIG. 2B is a photograph of a gel that shows the tissue distribution of cpg15 and cpg15-2 mRNA examined by reverse transcriptase-polymerase chain reaction (RT-PCR). FIG. 2C is a photograph of a gel that shows the distribution of cpg15 and cpg15-2 within the central nervous system. FIG. 2D is a photograph of a gel that shows the developmental expression of cpg15 and cpg15-2 mRNA in embryonic and postnatal mice by RT-PCR.

FIG. 3A is an autoradiograph showing the presence of CPG15-2 in both the cell lysate and in the culture supernatant. Cell lysate (cell) and culture supernatant (sup) from HEK293 cells stably transfected with FLAG-tagged cpg15, cpg15-2, or empty vector (−) were immunoprecipitated with an anti-FLAG antibody, then probed on a Western blot with an anti-FLAG antibody. FIG. 3B is a series of images showing that CPG 15-2 is a GPI-anchored membrane protein. HEK293 cells expressing FLAG-tagged CPG15 or CPG15-2 were immunostained with an anti-FLAG antibody under non-permeabilizing condition (red). EGFP (green) coexpressed from the same vector marks the cells. Both CPG15 and CPG15-2 show a punctate staining on the cell surface. Phospholipase C (PLC) treatment prior to immunostaining significantly reduces the surface staining of CPG15 and CPG15-2.

FIG. 3C is a photograph of two gels that shows CPG15 and CPG15-2 are glycoproteins. Immunoprecipitated CPG15 and CPG15-2 from transfected HEK293 cells were resolved run on SDS-PAGE and stained for glycoproteins. The same gel was restained with Coomasie brilliant blue (CBB). Horse radish peroxidase (HRP) and trypsin inhibitor (trypsin inh) are positive and negative controls, respectively, for glycoprotein staining. Both CPG15 and CPG15-2 are glycosylated.

FIG. 4A is a photograph of a gel that shows the formation of CPG 15 and CPG15-2 homodimers and heterodimers by transfection of HEK293T cells with cpg15, cpg15-2, or empty vector (pcDNA3). Whole cell lysates were resolved on SDS-PAGE under reducing or non-reducing conditions. FIG. 4B is an autoradiograph showing the formation of CPG15 and CPG15-2 homodimers and heterodimers by co-immunoprecipitation of CPG15 and CPG15-2 from HEK293T cells transfected with cpg15 and cpg15-2 each tagged with FLAG or poly-His. Cell lysates from transfected cells were immunoprecipitated with anti-FLAG antibody then analyzed on Western blot using anti-His antibodies.

FIGS. 5A and 5B are graphs that show promotion of neurite extension and branching in hippocampal explants by CPG15 and CPG15-2. Hippocampal explants were cocultured with control HEK293, CPG15-expressing, or CPG 15-2-expressing HEK293 cell aggregates for 60-72 hours, then fixed and immunostained for Neurofilament M to visualize the neuronal processes. FIG. 5A shows the quantification of average neurite length measured as tip distance from the explant. Explants cocultured with CPG 15 or CPG 15-2 expressing HEK293 cells have significantly longer neurites (*P<0.01). FIG. 5B shows the quantification of branching measured as number of branch tips per each neurite growing out of an explant. Explants cocultured with CPG15 or CPG15-2 expressing HEK293 cells have significantly more branch tips (*P<0.01).

FIG. 6A is a series of images showing the similar neurite extension and outgrowth effects of CPG15 and CPG15-2. Dissociated cortical neurons were plated on dishes coated with CPG15, CPG15-2, or control proteins, and imaged 24 hours later. Representative images of cortical neurons plated on BSA, CPG15, or CPG15-2 coated dishes. Scale bar: 100 μm. FIG. 6B is a graph showing the neurite length of neurons plated on dishes coated with indicated proteins at 10 ng/μl. Neurons plated on CPG15, CPG15-2, and both together had longer neurites than those plated on BSA or a negative control solution (P<0.05). FIG. 6C is a graph that shows the primary number of neurons plated on dishes coated with indicated proteins at 10 ng/μl. Neurons plated on CPG 15, CPG15-2, and both together had more primary neurites than those plated on BSA (P<0.05). FIG. 6D is a graph that shows the percentage of cells with neurites on dishes coated with indicated proteins at 10 ng/μl. Cells plated on CPG15, CPG 15-2, and both together had more neurite positive cells than those plated on BSA (P<0.05). FIG. 6E is a graph that shows the neurite length of neurons plated on different concentrations of CPG15 (open circle) or CPG15-2 (closed circle). CPG15-2 is more potent at lower concentrations. FIG. 6F is a graph showing a distribution plot of neurons with different neurite lengths. Percentage of neurons (ordinate) with neurites longer than a given length (abscissa) is plotted for each coating condition. Each protein was applied at 10 ng/μl. The curve for neurons plated on CPG15 (open circle), CPG15-2 (closed circle), and CPG15+CPG15-2 (open triangle) overlap and are greater than BSA (closed square).

FIG. 7A is a graph showing that CPG15 and CPG15-2 promote survival of dissociated cortical neurons. Neurons were plated on dishes coated with 10 ng/μl of CPG15 or CPG15-2 and the percentage of live cells was quantified 24 hours later. FIG. 7B is a graph showing the survival of neurons plated on different concentrations of CPG15 (open circle) or CPG15-2 (closed circle).

FIG. 8 shows a schematic of the starvation assay using primary hippocampal or cortical neurons.

### DETAILED DESCRIPTION

Many diseases result from increased cell death, including neurodegenerative conditions, cardiac conditions, muscle conditions, liver conditions, bone conditions, skin conditions, aging and aging-related conditions, and autoimmune diseases. There is a need for effective compounds that can promote cell survival and can therefore be used as therapies for the treatment of such diseases. We have discovered CPG15-2, a functional homolog of s-CPG 15, which can promote cell survival in hippocampal and cortical neurons. Cell death pathways are conserved among various types of cells; therefore, CPG15-2 can be used as a compound to promote cell survival in neurons, as well as additional cell types. We have also discovered that CPG15-2 can promote cell growth and differentiation and can therefore be used in applications such as promoting the differentiation of stem cells. In addition, inhibitors of CPG15-2 can be used to treat conditions which result from undesirable cell survival. Diseases that result from undesirable cell survival include any form of cancer, tumor-associated angiogenesis, and conditions resulting from hyperactivity of the immune system. By preventing or reducing the biological activity of CPG 15-2, inhibitors of CPG 15-2 can be used to promote apoptosis in cells.

### Preparation of Purified CPG15-2

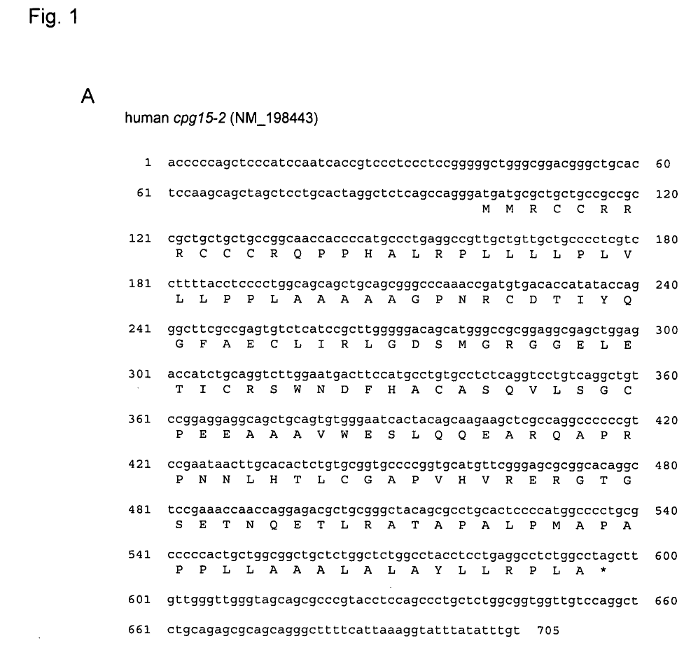

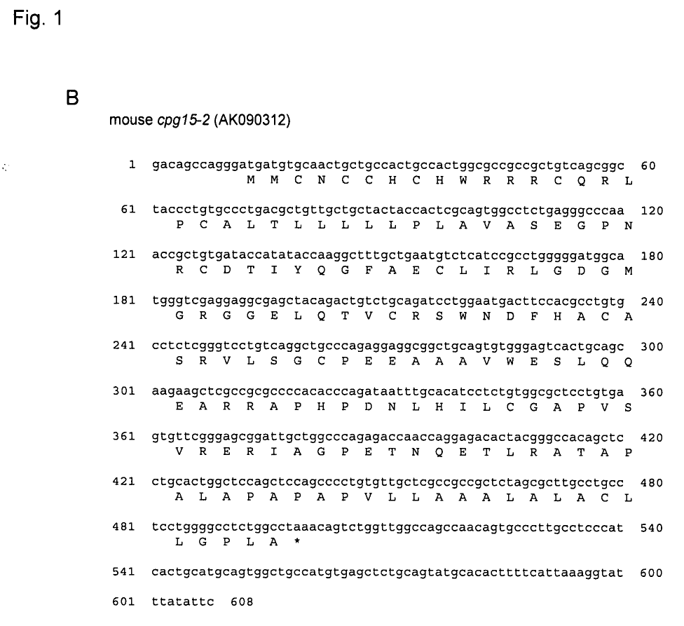

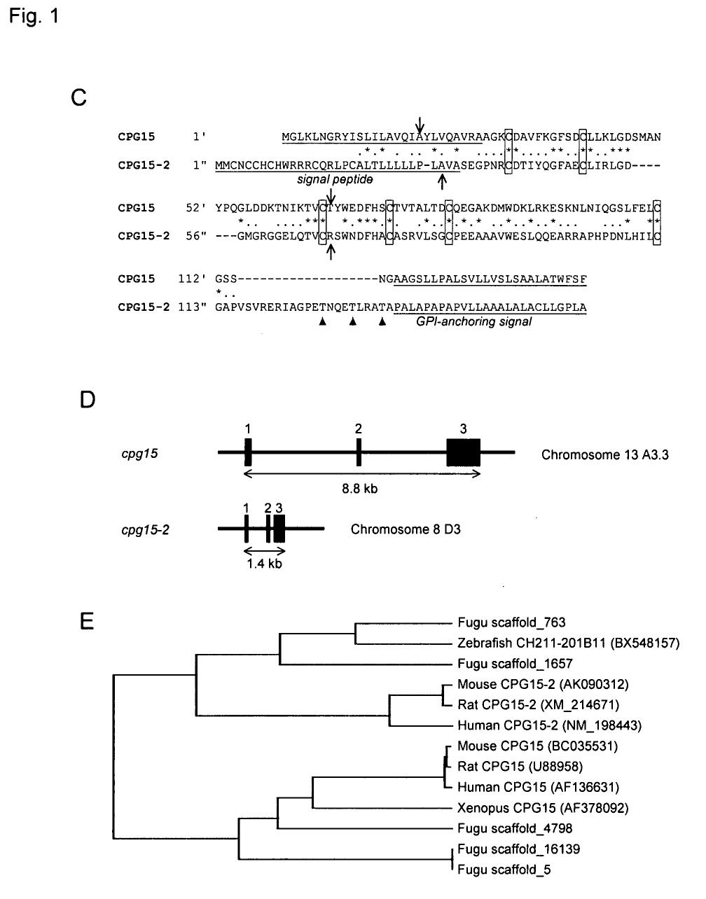

CPG 15-2 includes any amino acid sequence that is substantially identical to the to the amino acid sequences shown in FIGS. 1A and 1B and that encodes a protein that is capable of promoting survival or providing protection from cell death. CPG15-2 is also capable of promoting neuronal growth and differentiation. CPG 15-2 includes both the full length membrane bound form and the soluble secreted form of CPG15-2. Analogs or homologs of CPG15-2, which retain the biological activity of CPG15-2, are also included and can be constructed, for example, by making various substitutions of residues or sequences, deleting terminal or internal residues or sequences not needed for biological activity, or adding terminal or internal residues which may enhance biological activity. Amino acid substitutions, deletions, additions, or mutations can be made to improve expression, stability, or solubility of the protein in the various expression systems. Generally, substitutions are made conservatively and take into consideration the effect on biological activity. Mutations, deletions, or additions in nucleotide sequences constructed for expression of analog proteins or fragments thereof must, of course, preserve the reading frame of the coding sequences and preferably will not create complementary regions that could hybridize to produce secondary mRNA structures such as loops or hairpins which would adversely affect translation of the mRNA.

CPG15-2 analogs can also include any post-translationally modified forms. Examples of post-translational modifications include but are not limited to phosphorylation, glycosylation, hydroxylation, sulfation, acetylation, isoprenylation, proline isomerization, subunit dimerization or multimerization, and cross-linking or attachment to any other proteins, or fragments thereof, or membrane components, or fragments thereof (e.g., cleavage of the protein from the membrane with a membrane lipid component attached).

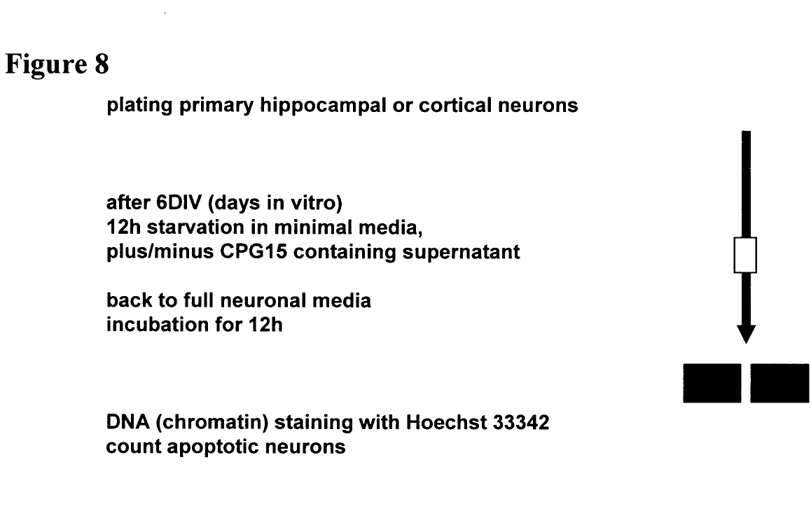

The biological activity of CPG 15-2 or any homologs, fragments, or mutants thereof can be determined, for example, by cell growth or cell death assays including but not limited to the serum starvation assays depicted in FIG. 8. In this assay, cells, preferably hippocampal cells, are grown in the absence of B27 (serum substitute; GIBCO-BRL) for a period of time long enough to initiate cell death. CPG15-2, or inhibitors or enhancers of CPG15-2 biological activity, are then added to the cells and the promotion of cell survival is measured by a reduction in the number of cells undergoing cell death. The biological activity of CPG 15-2 can also be measured, for example, by in vitro explant assays and neurite survival assays for process outgrowth such as those described herein and in Placzek et al., supra, Ringstedt et al., supra, Charron et al., supra, Wang et al., supra.

Desirably, the CPG 15-2 is preferably produced by recombinant DNA methods by inserting a DNA sequence encoding cpg15-2, homologs, fragments, or mutants thereof into a recombinant expression vector and expressing the DNA sequence under conditions promoting expression. General techniques for nucleic acid manipulation are described for example in Sambrook et al., Molecular Cloning: A Laboratory Manual, Vols. 1-3, Cold Spring Harbor Laboratory Press, 2 ed., 1989, or F. Ausubel et al., Current Protocols in Molecular Biology (Green Publishing and Wiley-Interscience:New York, 1987) and periodic updates. The DNA encoding CPG15-2 is operably linked to suitable transcriptional or translational regulatory elements derived from mammalian, viral, or insect genes. Such regulatory elements include a transcriptional promoter, an optional operator sequence to control transcription, a sequence encoding suitable mRNA ribosomal binding sites, and sequences which control the termination of transcription and translation. The ability to replicate in a host, usually conferred by an origin of replication, and a selection gene to facilitate recognition of transformants may additionally be incorporated.

Recombinant proteins can also be produced using methodologies for activating endogenous genes by positioning an exogenous regulatory sequence at various positions ranging from immediately adjacent to the gene of interest to 30 kilobases or further upstream of the transcribed region of an endogenous gene. Such methods are described, for example, in U.S. Pat. Nos. 5,641,670, 5,733,761, and 5,272071; WO 91/06666; WO 91/06667; and WO 90/11354, all of which are incorporated herein by reference.

The recombinant DNA can also include any type of protein tag sequence which may be useful for identifying the protein. Examples of protein tags include but are not limited to a polyhistidine tag, a FLAG tag, a myc tag, an HA tag, or a GST tag. The recombinant DNA can also encode for fusion proteins containing CPG15-2 fused with another protein. Preferred proteins include enzymatically active partners (e.g., for dye formation or substrate conversion) and fluorescent partners such as GFP, EGFB, and BFP.

The expression construct is introduced into the host cell using a method appropriate to the host cell, as will be apparent to one of skill in the art. The expression construct can be introduced for transient expression of the protein or stable expression by selecting cells using a selectable marker in order to generate a stable cell line that expresses the protein continuously. A variety of methods for introducing nucleic acids into host cells are known in the art, including, but not limited to, electroporation; transfection employing calcium chloride, rubidium chloride, calcium phosphate, DEAE-dextran, or other substances; microprojectile bombardment; lipofection; and infection (where the vector is an infectious agent).

Suitable host cells for expression of CPG 15-2 from recombinant vectors include prokaryotes, yeast, mammalian cells, insect cells, or amphibian cultured neurons under the control of appropriate promoters. Cell-free translation systems can also be employed to produce proteins using RNAs derived from the DNA constructs disclosed herein. Prokaryotes include gram negative or gram positive organisms, for example, E. coli or Bacillus spp. Prokaryotic expression hosts may be used for expression of CPG 15-2 or analogs thereof that do not require extensive proteolytic and disulfide processing. CPG15-2 may also be expressed in yeast, preferably from the Saccharomyces species, such as S. cerevisiae. Various mammalian or insect cell culture systems can also be employed to express recombinant protein. Baculovirus systems for production of heterologous proteins in insect cells are reviewed by Luckow and Summers, (Bio/Technology, 6:47, 1988). Examples of suitable mammalian host cell lines include endothelial cells, COS-7 monkey kidney cells, CV-1, L cells, C127, 3T3, Chinese hamster ovary (CHO), human embryonic kidney (HEK) cells, HeLa, 293, 293T, and BHK cell lines. Also, cell lines can be produced that over-express CPG15-2, allowing purification of CPG 15-2 proteins for biochemical characterization, large-scale production, antibody production, or patient therapy.

The proteins of the invention may be produced in vivo or in vitro, and may be chemically and/or enzymatically modified. Recombinant production not only offers a more economical strategy to produce the proteins of the invention, but also allows specific modification in the amino acid sequence and composition to tailor particular biochemical, catalytic and physical properties. For example, where increased solubility is desirable, one or more hydrophobic amino acids may be replaced with hydrophilic amino acids. Alternatively, where reduced or increased catalytic activity is required, one or more amino acids may be replaced or eliminated.

With respect to chemical and enzymatic modifications of contemplated proteins, many modifications are appropriate, including addition of mono-, and bifunctional linkers, coupling with protein and non-protein macromolecules, and glycosylation. For example, mono- and bifunctional linkers are especially advantageous where proteins are immobilized to a solid support, or covalently coupled to a molecule that enhances immunogenicity of contemplated proteins (e.g., KLH or BSA conjugation). Alternatively, the proteins may be coupled to antibodies or antibody fragments to allow rapid retrieval of the protein from a mixture of molecules. Further couplings include covalent and non-covalent coupling of proteins with molecules that prolong the serum half-life and/or reduce immunogenicity such as cyclodextranes and polyethylene glycols.

Purified CPG15-2 or analogs thereof are prepared by culturing suitable host/vector systems to express the recombinant proteins. Either the membrane bound version or the soluble version can be purified.

The same general methods are used for purification of the membrane bound and soluble forms of the protein. However, when the membrane bound protein is desired, the cells are solubilized first or a crude membrane extract is prepared to enrich for the membrane bound form.

In one example where the purification of soluble CPG15-2 is desired, the full-length CPG 15-2 is expressed, targeted to the membrane, via the secretion signal, where it is anchored, via the GPI anchor, and then cleaved off of the membrane to release the soluble form. The protein is likely to be released from the membrane with an additional membrane or protein component such as a membrane lipid. The protein is then purified from culture media or cell extracts.

In another example, supernatants from systems which secrete recombinant protein into culture media are first concentrated using a commercially available protein concentration filter, for example, an Amicon or Millipore Pellicon ultrafiltration unit.

Following the concentration step, the concentrate can be applied to a suitable purification matrix. For example, a suitable purification matrix can comprise a counter structure protein, lectin, or antibody molecule bound to a suitable support. Alternatively, an anion exchange resin can be employed, for example, a matrix or substrate having pendant diethylaminoethyl (DEAE) groups. The matrices can be acrylamide, agarose, dextran, cellulose, or other types of matrices commonly employed in protein purification. Alternatively, a cation exchange step can be employed. Suitable cation exchangers include various insoluble matrices comprising sulfopropyl or carboxymethyl groups. Gel filtration chromatography also provides a means of purifying the CPG15-2.

Affinity chromatography is a particularly preferred method of purifying CPG 15-2 and analogs thereof. For example, CPG15-2 expressed as a fusion protein comprising an immunoglobulin Fc region can be purified using Protein A or Protein G affinity chromatography. Monoclonal antibodies against the CPG15-2 protein may also be useful in affinity chromatography purification, by utilizing methods that are well-known in the art. A tagged version of the protein, such as a FLAG- or His-tagged version, can also be expressed and purified using antibodies directed to the tag sequence. In general, affinity chromatography will be performed using the soluble cellular fraction, or, in the case of tissue culture cells, the supernatant.

Finally, one or more reversed-phase high performance liquid chromatography (RP-HPLC) steps employing hydrophobic RP-HPLC media, e.g., silica gel having pendant methyl or other aliphatic groups, can be employed to further purify a CPG15-2 composition. Some or all of the foregoing purification steps, in various combinations, can also be employed to provide a homogeneous recombinant protein.

Recombinant protein produced in bacterial culture is usually isolated by initial extraction from cell pellets, followed by one or more concentration, salting-out, aqueous ion exchange or size exclusion chromatography steps. High performance liquid chromatography (HPLC) can be employed for final purification steps. Microbial cells employed in expression of recombinant protein can be disrupted by any convenient method, including freeze-thaw cycling, sonication, mechanical disruption, or use of cell lysing agents.

Fermentation of yeast which express CPG 15-2 greatly simplifies purification. Secreted recombinant protein resulting from a large-scale fermentation can be purified by methods analogous to those disclosed by Urdal et al. (J. Chromatog. 296:171, 1984). This reference describes two sequential, reversed-phase HPLC steps for purification of recombinant human GM-CSF on a preparative HPLC column.

Protein synthesized in recombinant culture is characterized by the presence of cell components, including proteins, in amounts and of a purity which depend upon the purification steps taken to recover the inventive protein from the culture. These components ordinarily will be of yeast, prokaryotic or non-human higher eukaryotic origin and preferably are present in innocuous contaminant quantities, on the order of less than about 10%, preferably less than 5%, 4%, 3%, 2%, and most preferably less than 1% by weight.

In addition to the methods employing recombinant DNA, CPG15-2 can be purified from sources that naturally produce the protein. Examples of these sources include neuronal cells and brain lysates isolated from mouse or rat brain after seizures. In particular, it is preferred that the brain lysate predominantly includes the hippocampus and the cerebral cortex. The CPG15-2 from these sources can be purified and concentrated using any of the methods described above.

After purification, CPG15-2 may be exchanged into different buffers and/or concentrated by any of a variety of methods known to the art, including, but not limited to, filtration and dialysis. The purified CPG15-2 is preferably at least 85% pure, more preferably at least 95% pure, and most preferably at least 98% pure. In preferred embodiments, the purified CPG15-2 includes less than 20%, preferably less than 10%, 5%, or 2%, and most preferably less than 1% CPG15 or s-CPG15. Regardless of the exact numerical value of the purity, the CPG15-2 is sufficiently pure for use as a pharmaceutical product.

CPG 15-2 proteins, particularly short fragments of the protein which retain CPG15-2 biological activity can also be produced by chemical synthesis (e.g., by the methods described in Solid Phase Peptide Synthesis, 2^{nd} ed., 1984, The Pierce Chemical Co., Rockford, Ill.). Modifications to the protein can also be produced by chemical synthesis.

### Treatment of Conditions Involving Inappropriate Cell Death

The present invention features methods of treating diseases that are caused by or that involve undesirable cell death. The delicate balance between cell growth and death allows for continuous remodeling and reshaping of cellular processes. When this balance is shifted, conditions that involve excess cell growth or cell death result.

In general, most diseases that are caused by inappropriate cell death are the result of the misregulation of apoptotic signaling proteins. Although our current understanding of apoptotic signaling pathways is far from complete, many of the key signaling proteins are known. For example, apoptosis can be triggered by extracellular toxins, calcium influx, lack of necessary growth factors, and activation of so called death receptors such as Fas and TNF-R. Downstream signaling proteins include Bcl-2 family members (Bcl-2, Bcl-X₁, Bax, Bad), proteins of the caspase/calpain family (activator caspases: 8,9,10, 12 and effector caspases: 3, 6, 7), Apaf-1, as well as several transcription factors such as p53 and c-jun. Any disease in which signaling proteins are deregulated such that they shift the balance in favor of increased apoptosis, is treatable by the methods provided herein.

It is a preferred embodiment of the invention that the methods or compositions comprising CPG15-2 be used to treat neurological conditions. CPG15-2 is useful in promoting the development, maintenance, or survival of neurons in vitro and in vivo, including central (brain and spinal chord), peripheral (sympathetic, parasympathetic, sensory, and enteric neurons), and motor neurons. Specific examples of neurological conditions include, but are not limited to, AD, PD, HD, stroke, ALS, peripheral neuropathies, trauma, and other conditions characterized by necrosis or loss of neurons. In addition, peripheral neuropathies associated with certain conditions, such as diabetes, AIDS, or chemotherapy can also be treated using the methods and compositions of the present invention.

Additional examples of neurological conditions considered treatable by the methods of the present invention include dementia with Lewy bodies (DLB), multiple system atrophy (MSA), muscular dystrophy, progressive supranuclear palsy (PSP), corticobasal degeneration, rare extrapyramidal conditions, multisystemic neuronal degeneration, synucleinopathies characterized by neuronal or glial inclusions of synuclein, Bell's palsy, Pick's disease, Kennedy disease, age-related conditions such as senility, Meniere's disease, multiple sclerosis (MS), spinocerebellar ataxia type I, spinobulbar muscular atrophy, and Machado-Joseph disease. The methods and compositions of the present invention can also be used to treat conditions of the retina and optic nerve, such as those characterized by an increase in cell death in the retinal cells, and preferably the retinal ganglion cells. Examples include retinal degeneration, retinitis pigmentosa, diabetic retinopathy, age-related macular degeneration and general macular dystrophies.

The methods of the present invention can also be used to treat non-neuronal conditions which are caused by an increase in cell death. Examples of additional conditions characterized by an increase in cell death include liver disease; pulmonary disease; conditions of the skin such as trauma or burn; conditions of the bone, muscle joint, or cartilage; cardiovascular diseases and conditions including, cardiac ischemia, congestive heart disease and myocardial infarction; autoimmune diseases such as rheumatoid arthritis; aging and aging related conditions; and immunodeficiency conditions such as those involving enhanced lymphocyte apoptosis.

Reagents that are used to treat conditions involving inappropriate cell death can include, without limitation, CPG 15-2 protein or fragments thereof, and any cpgl 5-2 nucleic acid including DNA, cDNA, RNA, mRNA. Delivery of the protein to the affected tissue can be accomplished using appropriate packaging or administration systems. Gene therapy can be used to deliver nucleic acid molecules to the cells in need of such therapy in a form in which they can be taken up by the cells so that sufficient levels of protein can be produced.

### Treatment of Conditions Involving Undesirable Cell Survival

The present invention features methods of treating conditions that involve undesirable cell survival. These conditions can result from a deregulation of signaling proteins such that they shift the balance towards a decrease or inhibition of cell death. Non-limiting examples of such diseases include any form of cancer in which cell growth is left unchecked, any type of undesirable or tumor-associated angiogenesis, and conditions resulting from hyperactivity of the immune system.

CPG15-2 functions both to promote cell survival by preventing or inhibiting cell death and to promote cell growth and differentiation in specific cell types. In vitro assays such as those described herein can be used with a particular cell type to determine the effect of CPG15-2 in that cell type. As the promotion of differentiation typically results in a cessation of cellular proliferation, CPG15-2 itself can be used in certain cell types to inhibit proliferation of the cell. Purified forms of CPG 15-2, as described above, can be used to treat diseases that involve undesirable cell survival such as cancer, undesirable or tumor-associated angiogenesis, and conditions resulting from hyperactivity of the immune system.

Alternatively, as CPG15-2 can prevent or inhibit cell death, inhibitors of CPG15-2 can also be used to promote or increase cell death. Examples of inhibitors of CPG15-2 include antisense nucleobase oligomers directed to CPG15-2, RNAi molecules directed to CPG 15-2, antibodies that specifically recognize CPG 15-2, and truncated or other dominant negative forms of CPG15-2 that can block the activity of CPG15-2.

Examples of cancers that can be treated by the methods and compositions of the present invention include bladder, blood, bone, brain, breast, cartilage, colon kidney, liver, lung, lymph node, nervous tissue, ovary, pancreatic, prostate, skeletal muscle, skin, spinal cord, spleen, stomach, testes, thymus, thyroid, trachea, urogenital tract, ureter, urethra, uterus, or vaginal cancer.

### Antisense Nucleobase Oligomers

The present invention features the use of antisense nucleobase oligomers to downregulate expression of cpg15-2 mRNA which will lead to a reduction in expression or biological activity of CPG15-2. By binding to the complementary nucleic acid sequence (the sense or coding strand), antisense nucleobase oligomers are able to inhibit protein expression presumably through the enzymatic cleavage of the RNA strand by RNAse H. Preferably the antisense nucleobase oligomer is capable of reducing CPG15-2 expression or biological activity, analogs, or fragments thereof, protein expression in a cell by at least 10% relative to cells treated with a control oligomer, more preferably 25%, and most preferably 50% or greater. Methods for selecting and preparing antisense nucleobase oligomers are well known in the art. Methods for assaying levels of protein expression are also well known in the art and include western blotting, immunoprecipitation, and ELISA.

### RNA Interference

The present invention also features the use of RNA interference (RNAi) to inhibit expression of cpg15-2 which will lead to a reduction in the expression of CPG15-2. RNA interference (RNAi) refers to a mechanism of post-transcriptional gene silencing (PTGS) in which double-stranded RNA (dsRNA) corresponding to a gene or mRNA of interest is introduced into an organism resulting in the degradation of the corresponding mRNA. In the RNAi reaction, both the sense and anti-sense strands of a dsRNA molecule are processed into small RNA fragments or segments ranging in length from 19 to 25 nucleotides (nt), preferably 21 to 23 nt, and having 2-nucleotide 3′ tails. These dsRNAs are known as “guide RNAs” or “short interfering RNAs” (siRNAs). siRNAs can also include short hairpin RNAs (shRNAs) in which both strands of an siRNA duplex are included within a single RNA molecule. Alternatively, synthetic dsRNAs, which are 19 to 25 nt in length, preferably 21 to 23 nt, and have 2-nucleotide 3′ tails, can be synthesized, purified and used in the reaction.

The siRNA duplexes then bind to a nuclease complex composed of proteins that target and destroy endogenous mRNAs having homology to the siRNA within the complex. Although the identity of the proteins within the complex remains unclear, the function of the complex is to target the homologous mRNA molecule through base pairing interactions between one of the siRNA strands and the endogenous mRNA. The mRNA is then cleaved approximately 12 nt from the 3′ terminus of the siRNA and degraded. In this manner, specific mRNAs can be targeted and degraded, thereby resulting in a loss of protein expression from the targeted mRNA.

The specific requirements and modifications of dsRNA are described in PCT Publication No. WO01/75164 (incorporated herein by reference). While dsRNA molecules can vary in length, it is preferable to use siRNA molecules which are 19- to 25-nt in length, most preferably 21- to 23-nucleotides in length, and which have characteristic 2- to 3-nucleotide 3′ overhanging ends typically either (2′-deoxy)thymidine or uracil. The siRNAs typically comprise a 3′ hydroxyl group. Single stranded siRNA as well as blunt ended forms of dsRNA can also be used. In order to further enhance the stability of the RNA, the 3′ overhangs can be stabilized against degradation. In one such embodiment, the RNA is stabilized by including purine nucleotides, such as adenosine or guanosine. Alternatively, substitution of pyrimidine nucleotides by modified analogs, e.g., substitution of uridine 2-nucleotide overhangs by (2′-deoxy)thymide is tolerated and does not affect the efficiency of RNAi. The absence of a 2′ hydroxyl group significantly enhances the nuclease resistance of the overhang in tissue culture medium.

siRNA can be prepared using any of the methods known in the art including those set forth in PCT Publication No. WO01/75164 or using standard procedures for in vitro transcription of RNA and dsRNA annealing procedures as described in Elbashir et al. (Genes & Dev., 15:188-200, 2001). Elbashir et al. describe the preparation of siRNAs by incubation of dsRNA that corresponds to a sequence of the target gene in a cell-free Drosophila lysate from syncytial blastoderm Drosophila embryos under conditions in which the dsRNA is processed to generate siRNAs of about 21 to about 23 nucleotides, which are then isolated using techniques known to those of skill in the art. For example, gel electrophoresis can be used to separate the 21 to 23 nt RNAs and the RNAs can then be eluted from the gel slices. In addition, chromatography (e.g., size exclusion chromatography), glycerol gradient centrifugation, and affinity purification with antibody can be used to isolate the 21 to 23 nt RNAs.

In the present invention, the dsRNA, or siRNA, is substantially complementary to at least a part of the mRNA sequence of a cpg15-2 mRNA and can reduce or inhibit the expression or biological activity of CPG 15-2. Desirably, the siRNA is 100% complementary to 18 to 25 consecutive nucleotides of CPG 15-2. Preferably, the decrease in CPG15-2 biological activity is at least 5% relative to cells treated with a control dsRNA, shRNA, or siRNA, more preferably at least 10%, 20%, or 25%, and most preferably at least 50%. Methods for assaying levels of protein expression are also well known in the art and include western blotting, immunoprecipitation, and ELISA. Methods for assaying CPG 15-2 biological activity include apoptosis assays, such as the serum starvation assay described herein, neurite outgrowth assays as described herein, and cell survival assays such as those described herein.

In the present invention, the nucleic acids used include any modification that enhances the stability or function of the nucleic acid in any way. Examples include modifications to the phosphate backbone, the internucleotide linkage, or to the sugar moiety and all of the modifications disclosed in U.S. Patent Publication Nos. 20030114412 (see for example paragraphs 27-45 of the publication) and 20030114407 (see for example paragraphs 35-52 of the publication).

### Antibodies

Antibodies that specifically bind to CPG15-2 can also be used to inhibit CPG15-2 biological activity and therefore to promote cell death. Such antibodies can be monoclonal or polyclonal and can include affinity-purified forms. When used in vivo for the treatment or prevention of conditions resulting from undesirable cell survival, the antibodies of the subject invention are administered to the subject in therapeutically effective amounts. Preferably, the antibodies are administered parenterally, intravenously by continuous infusion, intraventricularly in the brain, or intraocularly. The dose and dosage regimen depends upon the severity of the disease, and the overall health of the subject. The amount of antibody administered is typically in the range of about 0.01 to about 10 mg/kg of subject weight.

For parenteral administration, the antibodies are formulated in a unit dosage injectable form (solution, suspension, emulsion) in association with a pharmaceutically acceptable parenteral vehicle. Such vehicles are inherently nontoxic, and non-therapeutic. Examples of such vehicles are water, saline, Ringer's solution, dextrose solution, and 5% human serum albumin. Nonaqueous vehicles such as fixed oils and ethyl oleate may also be used. Liposomes may be used as carriers. The vehicle may contain minor amounts of additives such as substances that enhance isotonicity and chemical stability, e.g., buffers and preservatives. The antibodies typically are formulated in such vehicles at concentrations of about 1 mg/ml to 10 mg/ml.

### Inhibitory Forms of CPG15-2

Dominant negative or truncated forms of CPG 15-2 that can inhibit the biological activity of sCPG15-2 can also be used block cell survival or to promote cell death. Dominant negatives are often thought to act by either sequestering a functional form of the protein and rendering it non-functional or by binding to and blocking a receptor for the protein. One example of a truncated form of CPG15-2 is t-CPG15-2, which lacks the amino acids encoding the GPI linkage sequence. In general, t-CPG15-2 is expressed from an engineered construct containing the nucleic acid sequence encoding CPG 15-2 but lacking the nucleotides that encode the GPI linkage sequence. Such a truncated form of CPG 15-2 does not follow the GPI linkage pathway but is instead secreted directly out of the cell without membrane attachment or modifications associated with membrane attachment. t-CPG15-2 is believed to interact with CPG15-2 and inhibit its activity.

### Dosages and Therapeutic Uses

By “therapeutically effective dose” herein is meant a dose that produces the therapeutic effects for which it is administered. The exact dose will depend on the condition to be treated, and may be ascertained by one skilled in the art using known techniques. In general, the CPG115-2 protein is administered at about 0.01 μg/kg to about 50 mg/kg per day, preferably 0.01 mg/kg to about 30 mg/kg per day, most preferably 0.1 mg/kg to about 20 mg/kg per day. The CPG15-2 protein may be given daily (e.g., once, twice, three times, or four times daily) or less frequently (e.g., once every other day, once or twice weekly, or monthly). In addition, as is known in the art, adjustments for age as well as the body weight, general health, sex, diet, time of administration, drug interaction, and the severity of the disease may be necessary, and will be ascertainable with routine experimentation by those skilled in the art.

A “subject” for the purposes of the present invention includes humans and other animals, preferably warm-blooded mammals including mice, rats, guinea pigs, hamsters, rabbits, cats, dogs, goats, sheep, cows, or monkeys. Thus, the methods are applicable to both human therapy and veterinary applications.

CPG 15-2 can be administered in a variety of ways, e.g., those routes known for specific indications, including, but not limited to, topically, orally, subcutaneously, intravenously, intracerebrally, intranasally, transdermally, intraperitoneally, intramuscularly, intrapulmonary, vaginally, rectally, intraarterially, intralesionally, intraventricularly in the brain, or intraocularly. The CPG15-2 can be administered continuously by infusion into the fluid reservoirs of the CNS, although bolus injection is acceptable, using techniques well known in the art, such as pumps or implantation. Administration can be accomplished by a constant- or programmable-flow implantable pump or by periodic injections. Sustained release systems can also be used. Generally, where the condition permits, one should formulate and dose the CPG15-2 for site-specific delivery. Administration can be continuous or periodic.

Semipermeable, implantable membrane devices are useful as a means for delivering drugs in certain circumstances. For example, cells that secrete CPG15-2 can be encapsulated, and such devices can be implanted into a subject, for example, into the brain or spinal cord (CSF) of a subject suffering from Parkinson's Disease. See, U.S. Pat. Nos. 6,042,579; 4,892,538; 5,011,472; 5,106,627; PCT Applications WO 91/10425; 91/10470; Winn et al., (Exper. Neurology, 113:322-329, 1991); Aebischer et al., (Exper. Neurology, 111:269-275, 1991); and Tresco et al., (ASAIO, 38:17-23, 1992), each of which is herein incorporated by reference. The pharmaceutical compositions of the present invention comprise CPG 15-2 in a form suitable for administration to a subject. In the preferred embodiment, the pharmaceutical compositions are in a water soluble form, and may include such physiologically acceptable materials as carriers, excipients, stabilizers, buffers, salts, antioxidants, hydrophilic polymers, amino acids, carbohydrates, ionic or nonionic surfactants, and polyethylene or propylene glycol. The CPG15-2 may be in a time-release form for implantation, or may be entrapped in microcapsules using techniques well known in the art. Additional excipients useful for pharmaceutical compositions include any of those listed in U.S. Patent Application No. 20030176672, herein incorporated by reference.

Optionally, but preferably, the formulation contains a pharmaceutically acceptable salt, preferably sodium chloride, and preferably at about physiological concentrations. Optionally, the formulations of the invention can contain a pharmaceutically acceptable preservative. In some embodiments the preservative concentration ranges from 0.1 to 2.0%, typically v/v. Suitable preservatives include those known in the pharmaceutical arts. Benzyl alcohol, phenol, m-cresol, methylparaben, and propylparaben are preferred preservatives. Optionally, the formulations of the invention can include a pharmaceutically acceptable surfactant. Preferred surfactants are non-ionic detergents. Preferred surfactants include Tween 20 and pluronic acid (F68). Suitable surfactant concentrations are 0.005 to 0.02%.

The compositions, including lyophilized forms, are prepared in general by compounding the components using generally available pharmaceutical compounding techniques, known per se. Methods well known in the art for making formulations are found, for example, in “Remington: The Science and Practice of Pharmacy” (20^{th} ed., ed. A. R. Gennaro, 2000, Lippincott Williams & Wilkins, Philadelphia, Pa.). A particular method for preparing a pharmaceutical composition of CPG 15-2 hereof comprises employing purified (according to any standard protein purification scheme) CPG15-2, in any one of several known buffer exchange methods, such as gel filtration or dialysis.

The CPG15-2 can also be delivered via a nucleic acid encoding cpg15-2. The nucleic acid can be any nucleic acid (DNA or RNA) including genomic DNA, cDNA, and mRNA encoding any form of CPG15-2 shown to promote cell survival, reduce or prevent cell death, or promote cell differentiation. The nucleic acids of the present invention include any modification that enhances the stability or function of the nucleic acid in any way. Examples include modifications to the phosphate backbone, the internucleotide linkage, or to the sugar moiety.

To simplify the manipulation and handling of the nucleic acid encoding CPG 15-2, the nucleic acid is preferably inserted into a cassette where it is operably liked to a promoter. The promoter must be capable of driving expression of cpg15-2 in the desired target cell. Selection of the appropriate promoter and generation of the recombinant cpg15-2 expressing vector are techniques well known to one skilled in the art.

The nucleic acid can be introduced into the cells by any means appropriate for the vector employed. Many such methods are well known in the art (Sambrook et al., supra, and Watson et al., Recombinant DNA, Chapter 12, 2d edition, Scientific American Books, 1992). Examples of methods of gene delivery include liposome mediated transfection, electroporation, calcium phosphate/DEAE dextran methods, gene gun, and microinjection.

Gene delivery using viral vectors such as adenoviral, retroviral, lentiviral, or adeno-associated viral vectors can also be used. Numerous vectors useful for this purpose are generally known and have been described (Miller, Human Gene Therapy 15:14, 1990; Friedman, Science 244:1275-1281, 1989; Eglitis and Anderson, BioTechniques 6:608-614, 1988; Tolstoshev and Anderson, Current Opinion in Biotechnology 1:55-61, 1990; Sharp, The Lancet 337:1277-1278, 1991; Cornetta et al., Nucleic Acid Research and Molecular Biology 36:311-322, 1987; Anderson, Science 226:401-409, 1984; Moen, Blood Cells 17:407-416, 1991; Miller and Rosman, Biotechniques 7:980-990, 1989; Rosenberg et al., N. Engl. J. Med 323:370, 1990, Groves et al., Nature, 362:453-457, 1993; Horrelou et al., Neuron, 5:393-402, 1990; Jiao et al., Nature 362:450-453, 1993; Davidson et al., Nature Genetics 3:2219-2223, 1993; Rubinson et al., Nature Genetics 33, 401-406, 2003; U.S. Pat. Nos. 6,180,613; 6,410,010; 5,399,346 all hereby incorporated by reference). These vectors include adenoviral vectors and adeno-associated virus-derived vectors, retroviral vectors (e.g., Moloney Murine Leukemia virus based vectors, Spleen Necrosis Virus based vectors, Friend Murine Leukemia based vectors, lentivirus based vectors (Lois C. et al., Science, 295:868-872, 2002; Rubinson et al., supra), papova virus based vectors (e.g., SV40 viral vectors), Herpes-Virus based vectors, viral vectors that contain or display the Vesicular Stomatitis Virus G-glycoprotein Spike, Semliki-Forest virus based vectors, Hepadnavirus based vectors, and Baculovirus based vectors. Adenovirus, adeno-associated virus, and lentivirus are the preferred viral vectors for treatment of neurological conditions since they do not require recipient cells to be actively dividing.

In gene therapy applications, genes are introduced into cells in order to achieve in vivo synthesis of a therapeutically effective genetic product. “Gene therapy” includes both conventional gene therapy where a lasting effect is achieved by a single treatment, and the administration of gene therapeutic agents, which involves the one time or repeated administration of a therapeutically effective DNA or mRNA. Standard gene therapy methods typically allow for transient protein expression at the target site ranging from several hours to several weeks. Re-application of the nucleic acid can be utilized as needed to provide additional periods of expression of CPG15-2.

Any of the aforementioned delivery methods can also be used for delivery of nucleic acids including cpg15-2, antisense nucleobase oligomers, RNAi, dominant negative or truncated forms of CPG15-2, or antibodies directed to CPG15-2. The delivery of such CPG 15-2 inhibitors as therapeutic agents can be useful for the treatment of diseases characterized by inappropriate cell death or undesirable cell survival.

### In Vitro Uses

CPG15-2 can be used in a variety of in vitro applications. These applications include adding CPG 15-2 to cell culture media to promote the growth and survival of cells grown in culture. CPG 15-2 can also be used in stem cell growth applications where both the growth and survival promoting functions as well as the differentiating functions are useful. In addition, CPG15-2 can be used for applications relating to repairing and regenerating damaged tissue or organs by growing the tissue or organs ex vivo in the presence of CPG15-2. These examples are described in detail below.

### Cell Culture

CPG 15-2 is useful as a component of culture media for use in culturing cells in vitro or ex vivo. In one example the cells are cells of the nervous system. The CPG15-2 can be added to the media before or after the addition of the media to the cells. CPG 15-2 may also be added directly to the cells when needed or as a part of any other solution added to the cells. The amount of CPG15-2 added to the media is dependent on the type of cells used and the number of passages of the cells but can be determined empirically by the skilled artisan. Typically the amount of CPG15-2 added to the media will range from 0.001 μg/mL to 10 mg/mL, preferably 0.1 μg/mL to 1000 μg/mL, and most preferably 1.0 μg/mL to 100 μg/mL.

### Cell Growth and Differentiation

Differentiation of stem cells can be accomplished by exposing the cells to purified CPG15-2 protein or cpg15-2 nucleic acids using the methods described above. Stem cells can be proliferated in culture, and then differentiated in vitro or in situ into the cell types needed for therapy. Some of the progenitor or stem cell types which can be induced to differentiate using the purified CPG15-2 of the present invention include, embryonic stem cells, endothelial, muscle, nervous system (e.g., neural), pancreatic, bepatocyte, chondrocyte, cardiomyocyte, oligodendrocyte, and hematopoietic progenitor cells. Cells induced to differentiate into their specialized forms can then be used for therapeutic purposes. Stem cells induced to expand or differentiate in the presence of CPG 15-2 can be used, for example, for transplantation (e.g, organ, tissue, or bone marrow cell, or more specialized forms of transplantation where, for example, chondrocytes can be implanted into a joint surface defect in need of repair); the treatment of insulin dependent diabetes; the treatment of hematopoeitc conditions resulting from the loss of platelets or other hematopoietic cells; the treatment of cardiac injuries or liver injuries; and the treatment of neurodegenerative conditions such as epilepsy, stroke, ischemia, Huntington's disease, Parkinson's disease and Alzheimer's disease. Stem cells induced to differentiate using CPG 15-2 may also be appropriate for blood vessel repair or replacement. Stem cells induced to differentiate using CPG15-2 may also be appropriate for treating demyelinating conditions, such as Pelizaeus-Merzbacher disease, multiple sclerosis, leukodystrophies, neuritis and neuropathies. In one example, stem cells that have been induced to differentiate along a neuronal or myogenic lineage can be transplanted into the affected regions of a subject in need of cell replacement therapy.

Typically the amount of CPG15-2 added to the media will range from 0.001 μg/mL to 100 μg/mL. The CPG15-2 can also be attached to or mixed with a matrix for immobilization. CPG15-2 can also be administered to the patient after stem cell therapy or transplantation has been administered.

### Tissue or Organ Transplantation

CPG15-2 or nucleic acids encoding cpg15-2 can also be used to promote cell survival and/or differentiation for tissue and organ transplantation, the repair of diseased or damaged tissues and organs, and replacement tissue and organ engineering. The survival and differentiation promoting functions of CPG15-2 make this protein amenable as an added nutrient or type of growth factor in methods for sustaining organ or tissue survival in culture, e.g., prior to transplantation of the organ or tissue.

Desirably, the organ is a bladder, blood vessel, brain, nervous tissue, glial tissue, esophagus, fallopian tube, heart, pancreas, intestines, gallbladder, kidney, liver, lung, ovaries, prostate, spinal cord, spleen, stomach, testes, thymus, thyroid, trachea, urogenital tract, ureter, urethra, uterus, breast, skeletal muscle, skin, bone, or cartilage, or any part thereof of these organs. In desired embodiments, the tissue includes one or more cell-types derived from bladder, blood vessel, brain, nervous tissue, glial tissue, esophagus, fallopian tube, heart, pancreas, intestines, gallbladder, kidney, liver, lung, ovaries, prostate, spinal cord, spleen, stomach, testes, thymus, thyroid, trachea, urogenital tract, ureter, urethra, uterus, breast, skeletal muscle, skin, bone, or cartilage.

In addition, CPG 15-2 can be used to promote growth and differentiation in applications involving the growth of natural or synthetic tissues or organs in vitro. Tissue engineering is a method by which new living tissues are created in the laboratory to replace diseased or traumatized tissue. Methods for expanding various cell types used in tissue engineering are described in U.S. Pat. No. 6,582,960, herein incorporated by reference. In one example, CPG15-2 is added to a matrix or scaffold used for the growth of neuronal cells for central nervous system nerve regeneration. CPG15-2 can be perfused into any in vitro system used for the survival or promotion of tissue or organ growth. Administration of CPG15-2 can also be continued after the organ or tissue has been transplanted into the subject.

One particular strategy that has been created to regenerate new tissue is to (i) isolate specific cells from tissue; (ii) expand the isolated cells in vitro; and (iii) implant the expanded cells into the diseased or traumatized tissue so that the implanted cells proliferate in vivo and eventually replace or repair the tissue defect (Langer et al. Science 260:920-926, 1993). This technique has been applied to a variety of cell types and tissue defects (for example see Brittberg et al., N. Engl. J. Med., 331:889-895, 1994; Rheinwald et al., Cell, 6:331-344, 1975; Langer et al., supra). Isolated cells can be either differentiated cells from specific tissues or undifferentiated progenitor cells (stem cells). In both cases, establishment of appropriate culture conditions for cell expansion using CPG 15-2 is extremely important in order to maintain or improve their potential to regenerate structural and functional tissue equivalents (Rheinwald et al., supra).

According to the present invention, any cell type desirable for use in tissue engineering that can be isolated is used to regenerate tissue. Non-limiting examples include endothelial cells, muscle cells, skin cells, hepatocytes, chondrocytes, and melanocytes. Desirably, CPG15-2 is added in an amount sufficient to promote the expansion of the cells or the tissue while preserving the appropriate differentiation properties of the cells to ensure successful regeneration of high quality tissue or organ for implantation.

### Assays for Cell Death

There are many assays for cell death that are known to the skilled artisan. The assays differ depending on the type of cell death being detected and, in some cases, the cell types of interest. In an apoptotic cell, the cell membrane-bound apoptotic bodies are engulfed and degraded by a macrophage. The nuclear chromatin becomes pyknotic and condenses against the nuclear membrane. In contrast, necrosis involves only modest condensation of chromatin. One general method for distinguishing between a healthy, apoptotic, or a necrotic cell is through the use of Hoechst 33342 staining of the chromatin.

Some specific examples of assays for apoptosis and necrotic cell death are provided below. These examples are meant to illustrate the invention. They are not meant to limit the invention in any way.

### Assay for Necrotic Cell Death

Necrosis is a passive process in which collapse of internal homeostasis leads to cellular dissolution. The process involves loss of integrity of the plasma membrane and subsequent swelling, followed by lysis of the cell (Schwartz et al., Proc. Natl. Acad. Sci. USA, 90:980-984, 1993). Propidium iodide (PI) is known to bind to the DNA of cells undergoing primary and secondary necrosis (Vitale et al., Histochemistry, 100:223-229 1993). Necrotic cell death is characterized by loss of cell membrane integrity and permeability to dyes such as PI. Necrosis may be distinguished from apoptosis in that cell membranes remain intact in the early stages of apoptosis. As a consequence a dye exclusion assay using PI should be used in parallel with an assay for apoptosis, as described below in order to distinguish apoptotic from necrotic cell death, and the percentage of cells undergoing necrosis may be measured at various times before and after treatment with CPG15-2.

### Assay for Apoptotic Cell Death

Detection of programmed cell death or apoptosis may be accomplished using standard methods known to those in the art. The percentage of cells undergoing apoptosis may be measured at various times before and after treatment with CPG15-2 and compared with a control population of cells not treated with CPG15-2. The morphology of cells undergoing apoptotic cell death is characterized by a shrinking of the cell cytoplasm and nucleus, and condensation and fragmentation of the chromatin (Wyllie et al., J. Pathol. 142:67-77, 1984). One of the earliest events in programmed cell death is the translocation of phosphatidylserine, a membrane phospholipid from the inner side of the plasma membrane to the outer side. Annexin V is a calcium-dependent phospholipid binding protein that has a high affinity for membrane bound phosphatidylserine and thus annexin V-FITC can be used to stain cells undergoing apoptosis with detection and quantitation of apoptotic cells by flow cytometry or any other method of fluorescent detection (Vermes et al., J. Immunol. Methodol., 184:39-51, 1995; Walton et al., Neuroreport, 3:3871-3875, 1997). Accordingly, annexin V, when attached to a solid support such as a bead or a resin, can be used as an affinity ligand for binding apoptotic cells in solution. Similarly, annexin V is used as the basis for a fluorescent-activated cell sorting (FACS) separation process, another assay method well-known to the skilled artisan.

Additional assays for apoptosis in neuronal cells are disclosed by: Melino et al., Mol. Cell. Biol. 14:6584-6596, 1994; Rosenbaum et al., Ann. Neurol. 36:864-870, 1994; Sato et al., J. Neurobiol., 25:1227-1234, 1994; Ferrari et al., J. Neurosci., 1516:2857-2866, 1995; Talley et al., Mol. Cell. Biol. 1585:2359-2366, 1995; and Walkinshaw et al., J. Clin. Invest., 95:2458-2464, 1995; and U.S. Pat. Nos. 6,174,869 and 6,379,882, each of which is herein incorporated by reference.

### Assays for Cell Survival and Cell Proliferation

There are many standard assays for cell survival and proliferation known in the art. Examples of cell proliferation assays include BrdU labeling and cell counting experiments; quantitative assays for DNA synthesis such as ³H-thymidine incorporation. Cell survival can also be measured by trypan blue staining. Only non-viable cells absorb the trypan blue dye and appear blue. Cells stained with trypan blue can be counted using a hemocytometer to determine the number of non-viable and viable cells.

Cell survival can also be measured at various times before and after treatment with CPG 15-2 using the MTT assay. The MTT assay is a measure of mitochondrial activity in cells and is a general indicator of cell viability. The MTT assay is based on the ability of living cells to take in and process the dye known as MTT (3-(4,5-dimethylthiazol-2-yl)-2,5-diphenyl tetrazolium bromide; Sigma Chemical Co., St. Louis, Mo.), an active process which dead cells cannot complete. The assay is described in Mosmann et al. (J. Immunol. Meth. 65:55-63, 1983); Barres et al. (Cell 70:31-46, 1992); and Barres et al. (Development 118:283-295, 1993). MTT is added to the cell culture and incubated at 37° C. for one hour. Viable cells with active mitochondria cleave the tetrazolium ring into a visible dark blue formazan reaction product. Viable and dead cells are counted by bright field microscopy at various times, e.g., 24, 48, or 72 hours after treatment with s-CPG15-2.

### Interpretation of Results

Cell death can be evaluated using light microscopy following the staining of cells with the mitochondrial dye MTT, or by fluorescent/light microscopy following the staining of cells with propidium iodide (PI) or annexin V. Cell death is also evaluated by FACS analysis following staining with PI or annexin V. The percentage of apoptotic cells may be determined based on the percentage of annexin V positive cells that are not PI or MTT positive. However, there are some cells in later stages of apoptosis that also exhibit a loss of cell membrane integrity and stain positive with PI (i.e., they are undergoing secondary necrosis).

### Assays for Cell Differentiation

Cell differentiation can be measured by assays which quantitate the presence or absence of a defining characteristic or marker of a differentiated cell. In one example, stem cell conversion to neurons can be measured by expression of neuronal markers such as neurofilament-M, Map2, and neuron specific enolase. In another example, the clonogenic Colony Assay offered by Cambrex Corporation, can be used to determine differentiation of hematopoietic progenitor cells into myeloid (CFU-GM), erythroid (CFU-E, BFU-E), megakaryocyte (CFU-Meg), and mixed (myeloid and erythroid) colonies.

### Animal Models

The use of animals in medical research is a major way to increase our knowledge of the pathogenesis and alleviation of diseases in both animals and humans. Experiments on animals with induced diseases or conditions can be done under controlled conditions. Mechanisms relating to basic cellular processes such as cell division and apoptosis are highly conserved between species, particularly within mammals. A successful non-human animal model of neuronal cell death offers the prospect of understanding the origin and mechanisms of many neuronal conditions. Existing non-human animal models of neurological conditions can also be used to further explore therapies for neurological conditions. Non-human animals can include mice, rats, guinea pigs, hamsters, rabbits, cats, dogs, goats, sheep, cows, monkeys, or other mammals. Animal models can also be used to explore therapies for non-neuronal conditions.

Animal models of CPG 15-2 overproduction can be generated by integrating one or more cpg15-2 nucleic acid sequences into the genome of an animal, (e.g., a mouse) according to standard transgenic techniques. Moreover, the effect of cpg15-2 gene mutations (e.g., dominant gene mutations) can be studied using transgenic mice carrying mutated cpg15-2 transgenes or by introducing such mutations into the endogenous cpg15-2 gene, using standard homologous recombination techniques.

A replacement-type targeting vector, which can be used to create a knockout model, can be constructed using an isogenic genomic clone. The targeting vector can be introduced into a suitably-derived line of embryonic stem (ES) cells by electroporation to generate ES cell lines. To generate chimeric founder mice, the targeted cell lines are injected into a mouse blastula-stage embryo. Heterozygous offspring can be interbred to homozygosity. Cpg15-2 knockout mice provide a tool for studying the role of CPG15-2 protein and nucleic acid molecules in embryonic development and in disease. Moreover, such mice provide the means, in vivo, for testing therapeutic compounds for amelioration of diseases or conditions involving a CPG15-2 protein or nucleic acid molecule-dependent pathway.

Animal models can also include specific crosses of transgenic or knock out animals. For example, one animal model could be achieved by crossing a cpg15-2 transgenic animal with a separate animal model for a neurological condition. This type of cross could be very useful in determining the ability of CPG15-2 to rescue the defect causing the neurological condition.

Animals may be obtained from a variety of commercial sources, for example Charles River Laboratories, and housed under conditions of controlled environment and diet.

### Screen for Interacting Molecules

We describe here the ability of CPG15-2 to promote cell survival. Although we do not wish to be bound to a particular theory, it is likely that CPG15-2 functions to inhibit one or more signaling proteins that induce cell death. As many of these cell death pathways are commonly used by many different types of cells, CPG15-2 can therefore be used as a screening tool to identify interacting proteins that are important for the induction of cell death pathways.

It is also possible that CPG15-2 functions as a survival factor and may interact with a receptor or protein that is required to initiate cell survival. By using CPG15-2 as a screening tool, it may be possible to identify a protein or receptor important for cell survival. Modulation of such a protein or receptor through the use of agonists or antagonists could then be used to initiate or inhibit cell death pathways.

There are many types of screens for interacting proteins known in the art, all of which are included herein as screens for CPG15-2 interacting proteins. Some examples include affinity chromatography using immobilized CPG15-2, co-immunoprecipitations, and genetic screens, such as yeast two-hybrid screens, and variations thereof. In one example, a fusion construct encoding CPG 15-2 fused to alkaline phosphatase is used to detect binding to a tissue sample or a library (Flanagan J. G., Curr. Biol. 9:R469-470, 1999; Zhang et al., J. Neurosci. 16:7182-7192, 1996; Cheng et al., Cell 82:371-381, 1995; Cheng et al., Cell 79:157-168, 1994). This method can be used to identify tissues which contain potential CPG 15-2 interacting proteins and which can then be used to generate expression libraries for additional screening. In another example, affinity chromatography using purified CPG15-2 is employed. In this approach the CPG15-2 is expressed, purified, and immobilized using any number of art-known methods including direct immobilization of CPG15-2 to any type of resin (e.g., sepharose or cellulose beads), immobilization through a protein tag on the CPG15-2 such as GST or His tag interacting with an appropriate resin (e.g., glutathione sepharose or agarose for the GST tag and nickel sepharose or agarose for the His tag), or immobilization through an interaction with an anti-CPG15-2 antibody which is linked to beads or a resin. Immobilization of the purified CPG15-2 is preferably done under conditions that allow proteins associated with the CPG15-2 to remain associated with it. Such conditions may include the use of buffers that minimize interference with protein-protein interactions.

A test mixture is then mixed with the immobilized CPG15-2. The test mixture can be a cell lysate from any type of mammalian cell culture. Preferred cell types include 293, 293T, PC12 cells, HeLa, BHK, 3T3, HaK, or primary neuronal cells. In addition, tissue samples such as brain tissue samples can also be homogenized and used in the screen. The cell/tissue lysate can be unlabeled or radioactively labeled in order to easily identify interacting proteins. Any interacting proteins will be immobilized onto the CPG15-2 resin and the beads are then washed several times to remove any non-specific binding proteins. After washing, the CPG15-2 bound to the beads and any interacting proteins are incubated under denaturing conditions to release the proteins and the proteins are then separated by electrophoresis. Various types of protein gels can be used including sodium dodecyl sulfate-polyacrylamide gel electrophoresis (SDS-PAGE) followed by autoradiography for labeled cell lysates or SDS-PAGE followed by Coomassie blue staining or silver staining for unlabeled lysates. These protein-staining methods are standard, art-known techniques. Potential interacting proteins are purified and sequenced. Compounds isolated by this method (or any other appropriate method) may, if desired, be further purified (e.g., by high performance liquid chromatography). Compounds that are identified as binding to a protein of the invention with an affinity constant less than or equal to 10 mM are considered particularly useful in the invention.

Proteins that are identified as interacting proteins using any type of screen described above are then assayed for their ability to promote or inhibit apoptosis as measured by any standard assay such as those described herein, and can be used accordingly. For example, it is likely that CPG15-2 interacts with pro-apoptotic proteins to inhibit their function. Any pro-apoptotic proteins identified in this screen can then be used therapeutically to treat diseases which are a result of a decrease in or inhibition of apoptosis. The best example of such diseases includes any form of cancer in which cellular proliferation is uncontrolled. Treatment of cancer cells with any pro-apoptotic protein identified in this screen could induce apoptosis thereby reducing the proliferative capacity of these cells.

### Screening Assays for Compounds that Modulate the Expression or Activity of CPG15-2

As discussed above, we have discovered that the expression of CPG15-2 is useful in promoting cell survival and cell differentiation. Conversely, inhibition of cpgl 5-2 expression is useful in promoting cell death. Based on these discoveries, compositions of the invention are useful for the high-throughput low-cost screening of candidate compounds to identify those that modulate the expression or biological activity of CPG15-2 protein or nucleic acid for therapeutic purposes.

Any number of methods are available for carrying out screening assays to identify new candidate compounds that alter the expression of a CPG15-2 nucleic acid molecule or protein. In one working example, candidate compounds are added at varying concentrations to the culture medium of cultured cells expressing a cpg15-2 nucleic acid molecule. Gene expression is then measured, for example, by microarray analysis, northern blot analysis (Ausubel et al., supra), or RT-PCR, using any appropriate fragment prepared from the nucleic acid molecule as a hybridization probe. The level of gene expression in the presence of the candidate compound is compared to the level measured in a control culture medium lacking the candidate compound. A compound that promotes an alteration such as an increase or a decrease in the expression or biological activity of a cpg15-2 gene, nucleic acid molecule, or protein or a functional equivalent thereof, is considered useful in the invention; such a molecule may be used, for example, as a therapeutic to treat conditions.

In another working example, the effect of candidate compounds may be measured at the level of protein expression using the same general approach and standard immunological techniques, such as Western blotting or immunoprecipitation with an antibody specific for a CPG15-2 protein. For example, immunoassays may be used to detect or monitor the expression CPG15-2 in an organism or a cell line. Polyclonal or monoclonal antibodies (produced as described herein) may be used in any standard immunoassay format (e.g., ELISA, western blot, and immunoprecipitation) to measure the level of the protein. In some embodiments, a compound that promotes an alteration such as an increase in the expression or biological activity of CPG15-2 protein is considered particularly useful. Again, such a molecule may be used, for example, as a therapeutic for conditions of excessive cell death. A compound that promotes a decrease in the expression or biological activity of CPG 15-2 is considered useful, for example, as a therapeutic for conditions of undesirable cell proliferation.

The aforementioned cell growth and differentiation assays, as well as the apoptotic cell death assays, such as the hippocampal cell starvation assay described in Example 9, are also useful for assessing the ability of compounds (for example, organic compounds; small molecules; nucleic acid ligands such as DNA, RNA, or mixed nucleotide aptamers; ligands; synthetic chemicals; proteins; agonists; and antagonists) to modulate the ability of CPG 15-2 to rescue cells from apoptosis or to promote cell survival and differentiation. The method of screening may also involve high-throughput techniques employing standard computerized robotic and microtiter plates as is described below.

In one example, the method involves screening a library for therapeutically-active agents by employing, for example, the hippocampal starvation assays described herein. Based on our demonstration that CPG15-2 can prevent starvation-induced apoptosis, it will be readily understood that an agent which enhances the ability of CPG15-2 to prevent starvation-induced apoptosis could be used as an effective therapeutic agent in a subject suffering from a disease associated with inappropriate cell death.

Accordingly, the methods of the invention simplify the evaluation, identification, and development of active agents such as drugs for the treatment of conditions caused by excessive cell death.

In general, the chemical screening methods of the invention provide a straightforward means for selecting natural product extracts or agents of interest from a large population which are further evaluated and condensed to a few active and selective materials. Constituents of this pool are then purified and evaluated in the methods of the invention to determine their ability to modulate the cell-survival promoting activity of CPG15-2.

### Test Extracts and Agents

In general, novel drugs are identified from large libraries of both natural product or synthetic (or semi-synthetic) extracts or chemical libraries according to methods known in the art. The screening methods of the present invention are appropriate and useful for testing agents from a variety of sources for possible activity in vitro. The initial screens may be performed using a diverse library of agents, but the method is suitable for a variety of other compounds and compound libraries. Such compound libraries can be combinatorial libraries, natural product libraries, or other small molecule libraries. In addition, compounds from commercial sources can be tested, as well as commercially available analogs of identified inhibitors.

Virtually any number of chemical extracts or compounds known to those skilled in the art of drug discovery and development can be screened using the methods described herein. Examples of such extracts or compounds include, but are not limited to, plant-, fungal-, prokaryotic- or animal-based extracts, fermentation broths, and synthetic compounds, as well as modification of existing compounds. Numerous methods are also available for generating random or directed synthesis (e.g., semi-synthesis or total synthesis) of any number of chemical compounds, including, but not limited to, saccharide-, lipid-, peptide-, and nucleic acid-based compounds, including nucleic-acid ligands such as apatmers. Synthetic compound libraries are commercially available from, for example, Nanoscale Combinatorial Synthesis Inc., Mountain View, Calif., ChemDiv Inc., San Diego, Calif., Pharmacopeia Drug Discovery, Princeton, N.J., and ArQule Inc., Medford, Mass. Alternatively, libraries of natural compounds in the form of bacterial, fungal, plant, and animal extracts are commercially available from a number of sources, including Phytera Inc., Worcester, Mass. and Panlabs Inc., Bothell, Wash. In addition, natural and synthetically produced libraries are produced, if desired, according to methods known in the art, e.g., by standard extraction and fractionation methods. Devices for the preparation of combinatorial libraries are also commercially available, for example, Advanced ChemTech, Louisville, Ky. and Argonaut Technologies Inc., San Carlos, Calif. Furthermore, if desired, any library or compound is readily modified using standard chemical, physical, or biochemical methods.

When a crude extract is found to have activity that modulates CPG15-2 cell survival promoting activity in vitro, further fractionation of the positive lead extract is necessary to isolate chemical constituents responsible for the observed effect. Thus, the goal of the extraction, fractionation, and purification process is the careful characterization and identification of a chemical entity within the crude extract having activity that modulates the ability of CPG15-2 to promote cell survival. Methods of fractionation and purification of such heterogenous extracts are known in the art

Since many of the compounds that constitute currently available combinatorial and natural products libraries, as well as those found in natural products preparations, are not characterized, the screening methods of this invention provide novel compounds which are active as agonists or antagonists in the particular assays, in addition to identifying known compounds which are active in the screens. Therefore, this invention includes such novel compounds, as well as the use of both novel and known compounds in pharmaceutical compositions and methods of treating disease characterized by excessive cell death such as AD, PD, HD, and ALS.

### EXAMPLES

The features and other details of the invention will now be more particularly described and pointed out in the following examples describing preferred techniques and experimental results. These examples are provided for the purpose of illustrating the invention and should not be construed as limiting.

### Example 1

### Isolation and Sequence Analysis of cpg15-2

cpg15-2 was identified in a Genbank database search for genes encoding proteins similar to human CPG15/neuritin (accession number AF136631) using the BLASTP program (http://www.ncbi.nlm.nih.gov/blast/) with default settings. The search yielded a human predicted mRNA MRCC2446 (accession number NM_{—}198443) that we termed human cpg15-2 (FIG. 1A) and a mouse cDNA clone G630049C14 (accession number AK090312) we termed mouse cpg15-2 (FIG. 1B). The mouse clone shared 34% identity and 59% similarity at the amino acid level with mouse CPG15 (FIG. 1A). No other mouse sequence with significant similarity was found, suggesting that cpg15 and cpg15-2 are the only members of this gene family in mouse.

The mouse cpg15-2 cDNA was isolated from adult mouse brain RNA by RT-PCR. For RT-PCR, poly (A)+ RNA was reverse transcribed using the SuperScript first-strand synthesis system for RT-PCR (Invitrogen, Carlsbad, Calif.), and the coding region of the cpg15-2 cDNA was amplified by PCR with the following primers: 5′-CCGCTCGAGCCACCATGATGTGCAACTGCTGCCA-3′ (15-2-s1; SEQ ID NO: 7) and 5′-TCCCCGCGGTTAGGCCAGAGGCCCCAGG-3′ (15-2-as1; SEQ ID NO: 8) designed according to the mouse cDNA sequence. PCR was done using Pfu polymerase (Stratagene, La Jolla, Calif.) at 95° C. for 45 seconds, 55° C. for 45 seconds, and 72° C. for 3 minutes per cycle for 25 cycles. Amplified DNA was digested with XhoI and SacII, and cloned into a modified pcDNA3 vector (Invitrogen) in which the XbaI site was changed to a SacII site. Sequencing of the cloned PCR fragment was used to confirm its identity with the mouse cDNA G630049C14.

Protein sequence comparisons were done using the Genetyx-Mac program (Software Development, Tokyo, Japan). The SignalP program (http://www.cbs.dtu.dk/services/SignalP/) was used to identify the signal peptide sequence, and big-PI Predictor (http://mendel.imp.univie.ac.at/sat/gpi/gpi_server.html) and NetOGlyc/NetNGlyc (http://www.cbs.dtu.dk/services/) were used to identify the GPI-anchoring sequence and glycosylation site, respectively. Exon-intron structures of cpg15 and cpg15-2 genes were deduced from comparison of the cDNA sequences (BC035531, AK090312) and the genomic DNA sequences (NT_{—}039579 and NT_{—}078586).

The predicted CPG15-2 protein is 162 amino acids long, 20 amino acids longer than CPG15 largely due to an insertion near its carboxy (C) terminus (FIG. 1C). The two proteins share multiple structural features. As with CPG15, the CPG15-2 sequence predicts a signal peptide at the amino (N) terminus, suggesting that it may be secreted or membrane associated. CPG15-2 also has a potential glycosylphosphatidylinositol (GPI)-anchoring signal at its C-terminus like CPG15, suggesting that it may be attached to the membrane via a GPI anchor. The six cysteines of CPG15 are conserved in CPG15-2. All these similarities between CPG15 and CPG15-2 suggest that the two proteins are structurally conserved and undergo similar posttranslational processing. Three potential glycosylation sites were found in CPG15-2, but not in CPG15 (FIG. 1C).

cpg15 and cpg15-2 share a similar genomic structure (FIG. 1D), despite the divergence of their nucleotide sequence to a point where no similarity could be detected using the BLASTN program. The cpg15-2 gene is more compact than cpg15, with shorter introns. Yet, both genes contain three exons with exon/intron boundaries at similar positions in the corresponding protein sequence (FIG. 1C).

To examine the cross-species conservation of the cpg15 gene family, we searched for orthologues in other species. The human and rat genomes were found to contain one copy each of cpg15 and cpg15-2 (FIG. 1E). In human, an additional cDNA clone, DKFZp761P1315 (accession number AL390160), showed significant homology to CPG 15, but the homologous region was not part of the open reading frame, suggesting it may be a pseudogene. In pufferfish, Fugu rubripes, five candidate genes had significant similarity to CPG15 (FIG. 1E). Of these, three were more similar to CPG15, while two were similar to CPG15-2. It is not clear whether all five genes are expressed, since only partial sequence is available for some genes. Within the mammalian species, CPG15 was more highly conserved across species than CPG15-2 (FIG. 1E). No gene with significant homology was found in C. elegans or Drosophila, suggesting that the cpg15 family is unique to vertebrate species. Conservation of cpg15 and cpg15-2 orthologues across species and their similar genomic structure suggest that the two genes arose by a gene duplication event.

### Example 2

### Developmental and Tissue Specific Expression of cpg15-2 mRNA

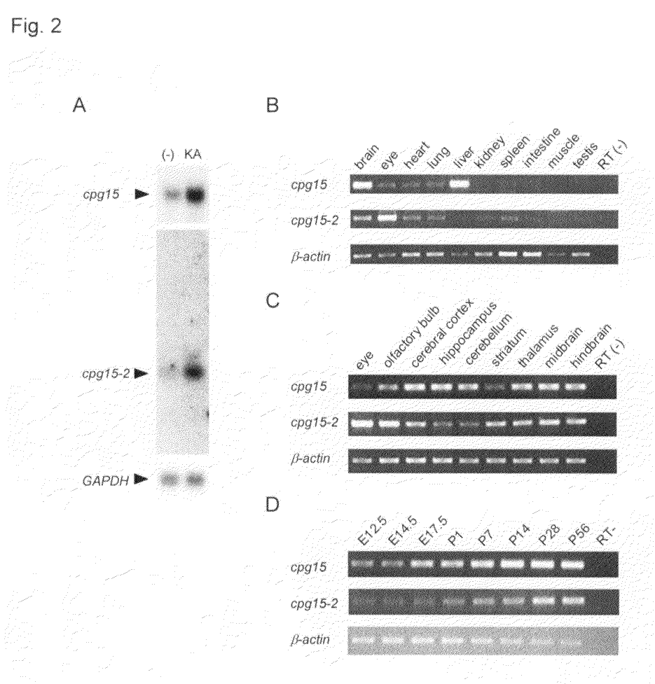

To being characterizing cpg15-2, we used Northern blot analysis to examine its expression in the mouse brain. For RNA preparation from kainate injected mice, cerebral cortices were harvested 6 hours after intraperitoneal injection of kainate (25 mg/kg) in PBS. Northern blot hybridization was done as described Sambrook et al., supra) with the following modifications. Poly (A)+ RNA selection was done using Oligotex (Qiagen, Valencia, Calif.). Ten micrograms of poly (A) enriched RNA was separated on 1% agarose gel containing formaldehyde, transferred to a nylon membrane, and hybridized with ³²P-labeled probes using stringent conditions. Probes were synthesized using the High Prime labeling kit (Roche, Indianapolis, Ind.) from the 1.6 kb mouse cpg15 cDNA fragment, 0.5-kb mouse cpg15-2 cDNA fragment or the 316-bp mouse GAPDH cDNA fragment excised from pTR1-GAPDH-mouse (Ambion, Austin, Tex.). The blot was hybridized first with the cpg15-2 probe and then reprobed for GAPDH (FIG. 2A).

We detected a faint 0.9 kb band, corresponding in size to the predicted cpg15-2 transcript (FIG. 2A). The signal intensity of the cpg15-2 band was significantly weaker than that of the cpg15 band, when probes of similar size and specific activity were applied to the same blot. Quantification of the bands indicates that cpg15-2 mRNA is approximately 60-fold less abundant in the brain than cpg15 mRNA. Since cpg15 was first characterized as an activity-regulated gene, we tested if cpg15-2 expression is also regulated by neural activity. We injected mice with kainate to induce massive neural activity and seizures, then compared cpg15 or cpg15-2 expression in brains of kainate-injected and uninjected control mice. The intensity of the band corresponding to cpg15 mRNA increased approximately three fold in response to kainate stimulation (FIG. 2A) (Fujino et al., Mol. Cell. Neurosci. 24:538-554, 2003). cpg15-2 mRNA showed a similar increase in response to kainate (FIG. 2A), suggesting that cpg15-2 is also an activity-regulated gene. In addition to the 0.9 kb band, a slightly larger band of 1.4 kb was detected with the cpg15-2 probe in the kainate-injected mice. This band may represent an alternatively spliced mRNA with different transcription initiation or termination sites, or the unspliced precursor RNA. Thus cpg15-2 is an activity-regulated gene expressed at lower levels than cpg15 in the brain.

We used RT-PCR to compare the tissue distribution of cpg15 and cpg15-2 mRNAs. The tissue specific and developmental expression of cpg15-2 mRNA was analyzed using RT-PCR (FIGS. 2B-2D). To prepare RNA, various tissues were dissected from C57BL/6 mice, frozen in liquid nitrogen, and kept at −80° C. until RNA extraction. For the developmental expression profile, RNA was prepared from whole heads at E12.5 and E14.5, and from brain at all other ages. Total RNA was extracted using the TRIZOL reagent (Invitrogen) according to the manufacturer's instructions. cDNA was synthesized from 2 μg of total RNA in a 20 μl reaction using the SuperScript first-strand synthesis system for RT-PCR (Invitrogen) with oligo (dT) priming. For each PCR, 0.5 μl of cDNA was used as template. PCR conditions were 94° C. for 30 seconds, 60° C. for 30 seconds, 72° C. for 1 minute per cycle, and were repeated for 21 cycles for cpg15, 26 cycles for cpg15-2, and 19 cycles for β-actin. Primer sequences were as follows:

(SEQ ID NO: 9)

5′-ACTCTCTCACTCTCTTTCTGTCTCTTCCTC-3′
and

(SEQ ID NO: 10)

5′-ACAGTCTGAAAAGCCCTTAAAGACTGCATC-3′
for cpg15;

(SEQ ID NO: 7)

5′-CCGCTCGAGCCACCATGATGTGCAACTGCTGCCA-3′
and

(SEQ ID NO: 8)

5′-TCCCCGCGGTTAGGCCAGAGGCCCCAGG-3′
for cpg15-2;

(SEQ ID NO: 11)

5′-TTGTAACCAACTGGGACGATATGG-3′
and

(SEQ ID NO: 12)

5′-GATCTTGATCTTCATGGTGCTAGG-3′
for β-actin.

PCR products were resolved on 2% agarose gel and visualized by ethidium bromide staining.

cpg15 mRNA was found to be most abundant in the brain and liver, whereas cpg15-2 mRNA was most abundant in the eye and brain (FIG. 2B). cpg15-2 mRNA was also detected at lower levels in the heart, lung, kidney, and spleen. Within the central nervous system, cpg15 and cpg15-2 were expressed in all regions examined, but the relative abundance of each mRNA across central nervous system regions was different (FIG. 2C). For example, cpg15-2 was expressed more abundantly in the eye and the olfactory bulb compared to the hippocampus and the cerebellum. In contrast, cpg15 was expressed more abundantly in the hippocampus and the cerebellum than in the eye and the olfactory bulb. Thus, cpg15 and cpg15-2 mRNAs are most abundant in the nervous system, and show largely overlapping but quantitatively different expression profiles. These results suggest that cpg15-2 may have a role in promoting cell survival and differentiation in any of these tissues.

We next examined cpg15 and cpg15-2 developmental expression profiles. Both cpg15 and cpg15-2 expression could be detected at E12.5, the earliest time tested, and gradually increased during embryonic and postnatal development (FIG. 2D). Increased cpg15 expression started around E17.5 and plateaued around P14. The Increase in cpg15-2 expression was more gradual, starting around P1 and plateauing later around P28. The different expression profiles suggest that cpg15 and cpg15-2 may play distinct roles in different central nervous system regions and at different developmental stages.

In summary, cpg15-2 is expressed primarily in the nervous system and regulated by neural activity like cpg15, but is expressed at lower levels and has different spatial and temporal expression profiles compared to cpg15.

### Example 3

### CPG15-2 is a Glycoprotein that Exists as a Both a Membrane Bound Form and a Secreted Soluble Form

To study the biochemical properties of CPG15 and CPG15-2 proteins, we made HEK293 cells stably expressing a FLAG-tagged CPG15 or CPG15-2. The FLAG-tagged proteins were then immunoprecipitated from the cell lysate and culture supernatant using an anti-FLAG antibody, then visualized on a Western blot using the same antibody.

The FLAG or poly-histidine (His) tagged constructs were generated as follows. A FLAG or poly-histidine (His) tag was inserted after the signal peptide sequence of CPG15 and CPG 15-2. An N-terminal fragment encoding the signal peptide and a tagged C-terminal fragment encoding the core domain and the GPI-anchoring signal were each generated by PCR from the full length cDNA with the following primers.

His-tagged cpg15 N-terminal fragment:

(SEQ ID NO: 13)

5′-GGAATTCGCCACCATGGGACTTAAGTTGAACGG-3′
and

(SEQ ID NO 14)

5′-GGGGTACCGCCTGCTGCTCTCACGG-3′;

His-tagged cpg15 C-terminal fragment:

(SEQ ID NO: 15)

5′-GGGGTACCCATCACCATCACCATCACAAGTGCGATGCAGTCTTT
AA-3′
and

(SEQ ID NO: 13)

5′-GGAATTCGCCACCATGGGACTTAAGTTGAACGG-3′;

FLAG-tagged cpg15-2 N-terminal fragment: 15-2-s1 and

(SEQ ID NO: 16)

5′-GGGGTACCGTTTGGGCCCTCAGAGGC-3′ (15-2-as2);

FLAG-tagged cpg15-2 C-terminal fragment:

(SEQ ID NO: 17)

5′GGGGTACCGACTATAAGGACGATGATGACAAGCGCTGTGATACCATAT
ACCAA-3′
and
15-2-as1;

His-tagged cpg15-2 N-terminal fragment:
15-2-s1 and 15-2-as2;
His-tagged cpg15-2 C-terminal fragment: 5′-

(SEQ ID NO: 18)

GGGGTACCCATCACCATCACCATCACGCAGGCCGCTGTGATACCATATAC
CAA-3′ (15-2-s2),
and
15-2-as1.

The N- and C-terminal fragments were digested with XhoI/KpnI and KpnI/SacII, respectively, then cloned into the pcDNA3 vector (Invitrogen). For viral expression, the FLAG-tagged cpg15 or cpg15-2 cDNAs were cloned into the BamHI site of the FUIGW lentiviral vector (Lois C. et al., Science 295:868-872 (2002)). Replication incompetent lentiviruses expressing tagged cpg15 and cpg15-2 were then generated as described (Lois et al., supra).

For generation of stable cell lines expressing FLAG-tagged CPG15 or CPG15-2, HEK293 cells were infected with a lentiviral vector coexpressing EGFP and CPG15, EGFP and CPG15-2, or EGFP only. Single cell clones expressing EGFP were isolated and expanded. Cells were harvested two days after transfection with one of the following methods. For whole cell extracts, cells were lysed in 100 μl of 1×SDS sample buffer per well, boiled for 5 minutes, then centrifuged at 14,000 rpm for 5 minutes to remove cell debris. For immunoprecipitation, culture supernatant and cell lysate were harvested separately. Culture supernatant was centrifuged at 3,000 rpm for 15 minutes to remove cells. Cells were lysed with RIPA114 buffer (50 mM Tris-HCl (pH 8.0), 100 mM NaCl, 5 mM EDTA, 1% Triton X-114, 0.2% SDS) and protease inhibitors (1% protease inhibitor cocktail, 1 mM PMSF; Sigma) for 1 hour on ice, then centrifuged at 14,000 rpm for 15 minutes at 4° C. to remove cell debris. Culture supernatant and cell lysate were each incubated with 2 μl (packed volume) of anti-FLAG M2 affinity gel (Sigma) or 0.3 μL of mouse anti-His monoclonal antibody (Sigma) and incubated overnight at 4° C. For anti-His antibody, protein-A agarose (Sigma) was added the following day and incubated further for 1 hour at 4° C. Immunoprecipitates were washed two times each with RIPA114 buffer and PBS, then boiled for 5 minutes in 25 μl of 1×SDS Sample buffer. Ten microliters were used for Western blot analysis.

Western blot analysis was done as described in Sambrook et al., (Molecular Cloning: A Laboratory Manual, Cold Spring Harbor Laboratory Press, New York) with the following modifications. Samples were subjected to 15% SDS-polyacrylamide gel electrophoresis then transferred on a nitrocellulose membrane (Schleicher & Schuell, Keene, N.H.) using semi-dry electroblotting apparatus (Biorad, Hercules, Calif.) at 10 V for 1 hour. After blocking the membrane with 10% skim milk, the membrane was incubated with mouse anti-FLAG monoclonal antibody (M2, 1:1000, Sigma), mouse anti-HA monoclonal antibody (1:000, Sigma) or mouse anti-His monoclonal antibody (1:1000, Sigma) for 1 hour at room temperature, then with HRP-conjugated anti-mouse IgG antibody (1:5000, Jackson Immuno Research, West Grove, Pa.) for 1 hour at room temperature. Bands were visualized with chemiluminescence (ECL, Amersham, Piscataway, N.J.).

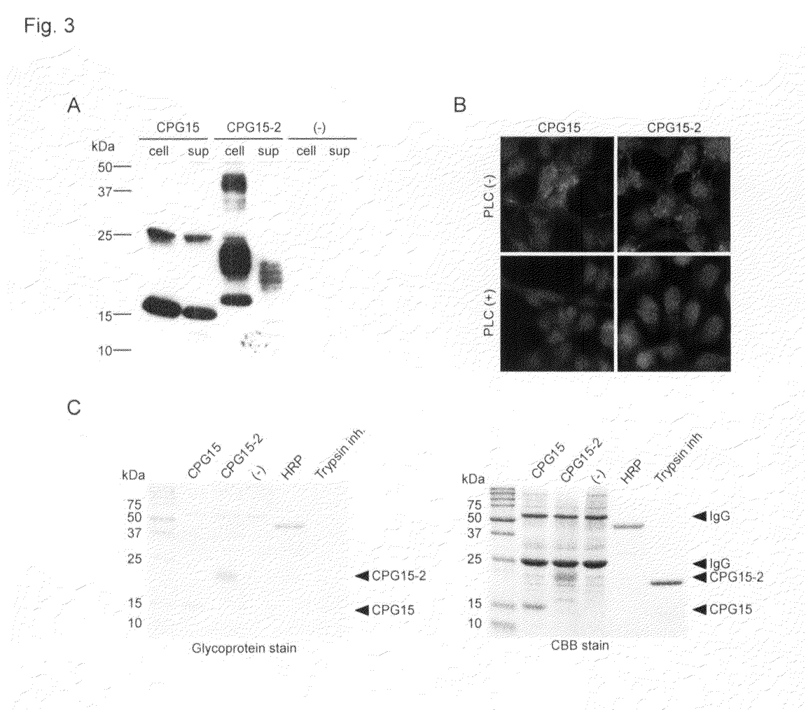

FLAG-tagged CPG15 purified from the cell lysate fraction migrated as 15 kDa and 25 kDa bands, with the 15 kDa band being more abundant (FIG. 3A). Both bands are larger than 10.8 kDa, the predicted size of the FLAG-tagged CPG 15 core domain after cleavage of the signal sequence and the GPI-anchoring signal, suggesting that CPG15 is post-translationally modified. The 15 kDa and 25 kDa bands likely represent monomeric and dimeric forms of CPG15, respectively (see below). Bands of similar mobility and intensity were detected in the supernatant fraction indicating that CPG 15 is secreted from the cells as previously reported (Putz, submitted Since the CPG15 in the supernatant fraction was not significantly different from the CPG 15 in the cell lysate, it is likely that either CPG15 is processed into the soluble form within the cell, or CPG15 is cleaved close to the membrane retaining most of the GPI molecule, resulting in a soluble CPG15 protein with similar molecular weight as the membrane-bound form.

FLAG-tagged CPG 15-2 in the cell lysate fraction migrated as a 17 kDa band and multiple bands around 20-22 kDa and 39-44 kDa, the 20-22 kDa bands being the most abundant. The predicted size of the FLAG-tagged CPG15-2 core domain is 12.5 kDa, approximately 2 kDa larger than that of FLAG-tagged CPG15. The 17 kDa band may be processed similarly to the 15 kDa band of CPG15. However, since the majority of CPG15-2 from the cell lysate migrated at a larger size in multiple bands, CPG15-2 likely undergoes additional modification compared to CPG15, possibly glycosylation. The 39-44 kDa bands likely correspond to the dimeric form of 20-22 kDa bands (see below).

FLAG-tagged CPG15-2 was also detected in the culture supernatant, indicating that there is a soluble secreted form of CPG15-2, like CPG15. The soluble CPG15-2 migrated as multiple 19-21 kDa bands, similar in appearance to the 20-22 kDa bands in the cell lysate fraction, but about 1 kDa smaller and less abundant. This is in contrast to CPG15 in the supernatant, which migrated at a similar size and intensity as the CPG15 from the cell lysate fraction. These results suggest that although CPG15 and CPG15-2 are both secreted, CPG 15-2 is less efficiently secreted and may be produced through a different type of processing compared to CPG15.

To determine if CPG15-2 is attached to the cell surface by a GPI-anchor as been shown for CPG15 (Naeve et al., Proc. Natl. Acad. Sci. USA 94:2648-2653, (1997)), we immunostained HEK293 cells expressing FLAG-tagged CPG15 or CPG15-2 with an anti-FLAG antibody. HEK293T cells were plated on coverslips coated with 0.5 mg/ml poly-L lysine at 3×10⁵ cells per well of 6-well plates. HEK293T cells were transfected the following day with 1 μg of the appropriate expression vector (FLAG-cpg15 or FLAG-cpg15-2) using Lipofectamine 2000 (Invitrogen). Phophatidylinositol-specific phospholipase C (Sigma) was added to the media of selected cells at 1 U/ml and incubated for 4 hours. Mouse anti-FLAG monoclonal antibody (M2, 1:1000, Sigma) was added directly to the media and incubated for 30 minutes at 37° C. Two days after transfection, cells were fixed with 4% formaldehyde for 15 minutes at 4° C., blocked for 1 hour with 10% goat serum in PBS, incubated with mouse anti-FLAG monoclonal antibody (M2, 1:1000, Sigma) for 1 hour at room temperature, then incubated with Rhodamine-conjugated goat anti-mouse IgG antibody (1:500, Jackson Immuno Research). Images were acquired with an epifluorescence microscope (Nikon, Tokyo, Japan) equipped with a digital camera (Spot2, Diagnostic Instruments, Sterling Heights, Mich.).

Under non-permeabilizing staining conditions, both proteins showed a similar punctate membrane staining (FIG. 3B), indicating that they are present on the outer cell surface. Addition of phospholipase C which cleaves the GPI-anchor significantly reduced the surface staining of both CPG15 and CPG15-2, suggesting that CPG15-2 is a GPI-anchored protein.

Since both CPG15 and CPG15-2 migrated at a size larger than predicted by their sequence, we tested whether they are glycosylated. Both CPG15 and CPG15-2 immunoprecipitated from the cell lysate fraction could be stained with a glycoprotein stain (FIG. 3C), indicating that both proteins are glycosylated. CPG15-2 glycoprotein staining for was stronger than CPG15, likely due to multiple glycosylation sites.

Taken together, these results indicate that both CPG15 and CPG15-2 are glycoproteins that exist in a GPI-anchored form on the cell surface and a soluble secreted form. As compared to CPG15, CPG15-2 appears more heavily glycosylated and less efficiently secreted.

### Example 4

### CPG15-2 Form Both Homodimers and Heterodimers with CPG15

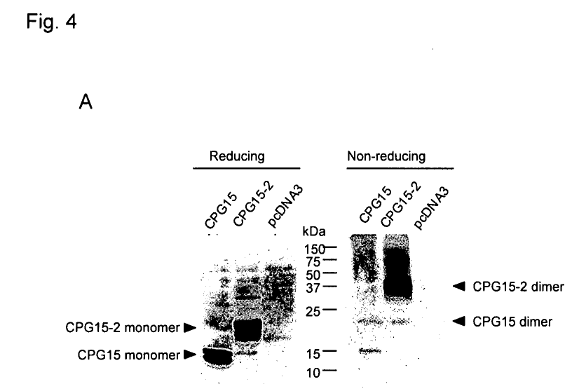

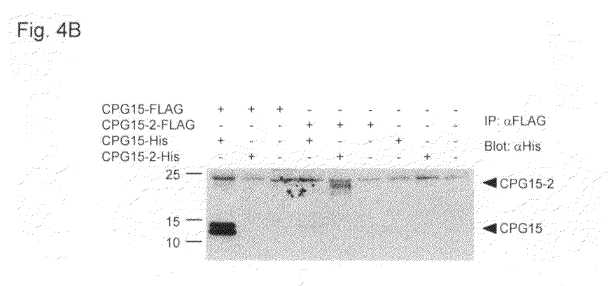

In order to determine if CPG 15-2 could form homodimers or heterodimers with CPG15 we analyzed whole cell extracts of HEK293T cells transfected with cpg15, cpg15-2, or empty vector (pcDNA3). HEK293T cells were cultured in DMEM (BioWhittaker) with 10% fetal bovine serum (BioWhittaker) and plated at 1×10⁶ cells per well in 6-well plates and transfected on the following day with 2 μg of DNA by Lipofectamine 2000 (Invitrogen). Cells were then harvested as described in Example 3 by SDS-PAGE under reducing or non-reducing conditions. Under non-reducing condition, bands with a molecular weight appropriate for dimer formation were observed (FIG. 4A) suggesting that CPG15 and CPG15-2 homodimers and heterodimers exist.

On Western blots, purified CPG 15 and CPG 15-2 showed high molecular weight bands suggestive of dimer formation (FIG. 3A). We performed co-immunoprecipitation experiment to examine if CPG15 and CPG15-2 form homodimers and perhaps heterodimers. We found that poly-histidine (His)-tagged CPG15 coimmunoprecipitated with FLAG-tagged CPG15 (FIG. 4B), confirming the ability of CPG15 to homodimerize. Similarly, His-tagged CPG15-2 coimmunoprecipitated with FLAG-tagged CPG15-2, demonstrating the same for CPG15-2. Interestingly, His-tagged CPG 15 coimmunoprecipitated with FLAG-tagged CPG 15-2, indicating that CPG 15 and CPG15-2 could also heterodimerize. The amount of CPG15 that coprecipitated with CPG15-2 was much less than the amount that coprecipitated with CPG15, suggesting that CPG15 form homodimers more efficiently than heterodimers with CPG15-2. Same was true for CPG15-2. Thus, both CPG15 and CPG15-2 form homodimers, and can also interact with each other at a lower affinity.

### Example 5

### CPG15-2 Protein Purification

We generated stable HEK293 lines expressing cpg15, cpg15-2, or EGFP as a source for protein purification. To generate stable cell lines, HEK293 cells were infected with Lentivirus coexpressing cpg15 or cpg15-2 with EGFP, or expressing only EGFP. Single cell clones expressing EGFP were isolated and expanded. Cells were plated on two to four 15 cm dishes with DMEM (BioWhittaker, Walkersville, Md.) supplemented with 10% FBS (BioWhittaker, Walkersville, Md.). After three to four days of culture, culture supernatant was collected, cleared by centrifugation at 3,000 rpm for 15 minutes, then incubated with 10 μl (packed volume) of anti-FLAG M2 affinity gel (Sigma) overnight at 4° C. Immunoprecipitate was washed five times with PBS, then eluted with 100 μl of 0.1 μg/μl 3×FLAG peptide (Sigma) in PBS supplemented with 0.02% BSA (New England Biolabs, Beverly, Mass.). Protein concentration was determined on a Western blot by silver staining (Pierce).

### Example 6

### CPG15 and CPG15-2 Promote Neurite Extension and Branching in Hippocampal Explants

Conservation of many of the biochemical properties between CPG 15 and CPG 15-2 suggested that their functional properties might also be similar. To test this hypothesis, brains were removed from postnatal day 3-5 Sprague-Dawley rats, and hippocampi isolated using a fine tungsten needle knife then further trimmed into 100-300 μm pieces. Isolated explants were embedded in a 4:3:1 mixture of rat tail collagen, matrigel (Collaborative Research) and DMEM together with cell aggregates at a distance ranging from 100-400 μm (Zhu et al., Neuron 23:473-485 (1999)). Cell aggregates were prepared by the hanging-drop method (Fan et al., Cell 79:1175-1186 (1994)). After collagen matrices were solidified, they were cultured in DMEM with 10% FBS and 100 μg/ml of penicillin and streptomycin at 37° C. in an incubator with 5% CO₂ for 60-72 hours. Explants were then fixed and stained for immunocytochemistry with an anti-Neurofilament M rabbit polyclonal antibody (Chemicon) to visualize neuronal processes (Li et al., Cell 96:807-818 (1999)).

Explants were imaged using a Nikon Eclipse E600Fn confocal microscopy system (Nikon) equipped with an Argon-HeNe laser. Images were acquired with Simple PCI software (version 3.5.0.1309, Compix Inc. Image system) and analyzed using Object-Image (Norbert Vischer) software for process tracing with Morphometry Macros (Edward Ruthazer, Cline lab) (http://www.cshl.org/labs/cline/morphometry.html). Number of tips per primary neurite was calculated as total number of tips per explant divided by total primary neurites per explant. Average neurite length was calculated as total neurite length (μm) per explant divided by total number of primary neurites per explant. Mean and standard error of mean (SEM) were calculated from the average value of 5-8 explants per condition. Statistical significance was determined by analysis of variance (ANOVA) and Student-Newman-Keuls (SNK) post hoc analysis using StatView software (SAS Institute).

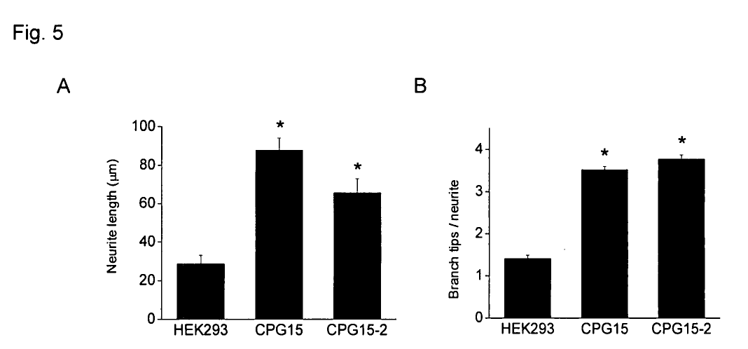

To test the effect of CPG 15 and CPG 15-2 on neurite growth, hippocampal explants were co-cultured with CPG 15 or CPG 15-2 expressing HEK293 cell aggregates in a collagen/matrigel matrix. After 60 to 72 hrs, the neurites growing out of the explant were measured for length and branching. When explants were co-cultured with CPG 15 or CPG15-2 expressing HEK293 cells, their neurites were significantly longer than those from explants co-cultured with control HEK293 cells (FIG. 5A). Explants co-cultured with CPG15 or CPG15-2 expressing HEK293 cells also had significantly more branch tips per neurite as compared to control explants (FIG. 5B), indicating that both CPG15 and CPG15-2 both promote neurite growth and branching.

### Example 7

### CPG15 and CPG15-2 Show Similar Efficacy in Promoting Neurite Extension and Outgrowth

To quantitatively compare the growth promoting functions of CPG15 and CPG15-2, we performed the neurite outgrowth assay on dissociated cortical neurons. Neurite outgrowth assays were done as described (Lemmon et al., Neuron 2:1597-1603 (1989) and Nakashiba et al., Mech Dev 111:47-60 (2002)). Purified protein (18 μl) of indicated concentration was applied in a 50 mm² circle on nitrocellulose coated 6-well dishes and incubated for 1 hour. Approximately 80% of protein was absorbed to the dish under these conditions. The dish was then blocked with 10 mg/ml BSA for 30 minutes. Dissociated cortical neurons were prepared as described (Fujino et al., Mol Cell Neurosci 24:538-554 (2003)) from cerebral cortices of E19 Sprague-Dawley rats and plated at 1.5×10⁵ cells per well. Cells were imaged after 24 hours using phase contrast microscopy. Neurite length was measured as the direct distance between the center of the soma and the tip of its longest neurite. Counting was done blind to experiment al conditions. Mean and SEM were calculated from 76-111 neurons for each condition.

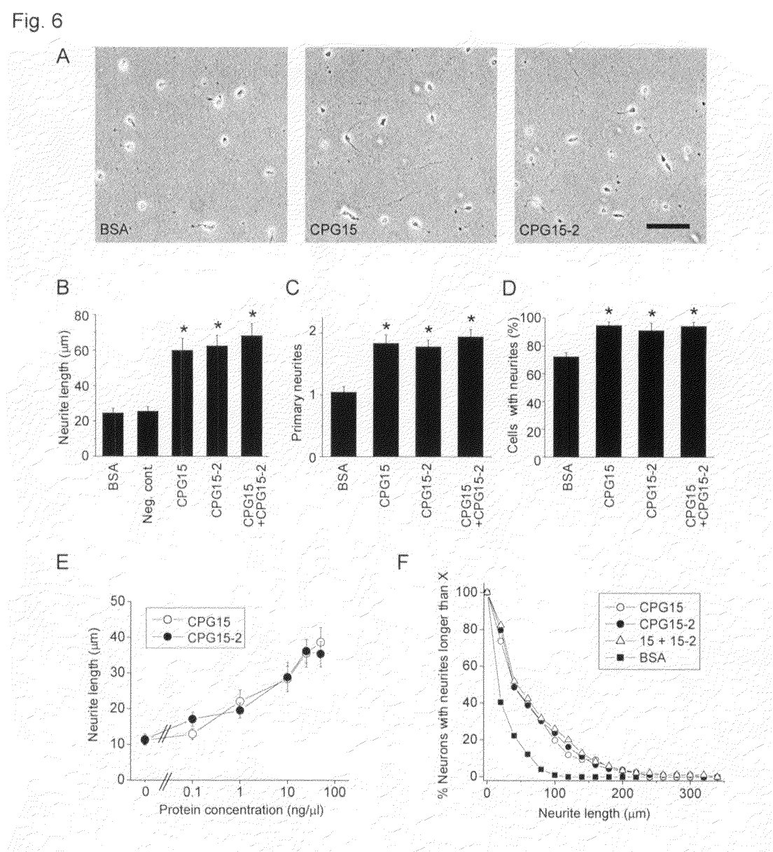

Dissociated neurons were plated on dishes coated with purified CPG15, CPG15-2, or control proteins, and neurite growth was assayed 24 hours later. Neurons plated on CPG 15 or CPG 15-2 coated dishes had significantly longer neurites than those plated on BSA coated dishes (FIGS. 6A and 6B), confirming the neurite growth effect of these proteins observed in the hippocampal explant assay. No effect on neurite growth was observed when dishes were coated with a control solution purified from the supernatant of control HEK293 cells (FIG. 6B), confirming that the positive growth effect of CPG15 and CPG15-2 is not due to contaminating material from the HEK293 cells or the culture media. We also examined if CPG15 and CPG15-2 affect initial neurite outgrowth by counting the number of primary neurites for each neuron. Neurons plated on CPG15 or CPG15-2 coated dishes had significantly more primary neurites compared to those plated on BSA coated dishes (FIG. 6C). The percentage of cells with neurites increased from 72% to over 90% (FIG. 6D).

When we compared the average branch tip number for each primary neurite, we found that on average, there was less than one branch point per neurite, and CPG 15 and CPG 15-2 did not significantly increase branch tip number. The difference in the branching effect observed between the hippocampal explant assay and the cortical cultures could be due to the shorter culture period (24 hours) for the cortical cultures compared to the hippocampal explants (60-72 hours).

We compared the efficacy of CPG 15 and CPG 15-2 by plating dishes with various concentration of the two proteins. Neurons showed a similar dose response curve for each protein (FIG. 6E), with growth promoting effects observed at concentrations above 1 ng/μl and saturating around 25 ng/μl. No significant increase was observed in neurite length, number of primary neurites, and percentage of cells with neurites when dishes were coated with both CPG15 and CPG15-2 (FIGS. 6B, 6C, 6D), demonstrating that the effect of the two proteins is not additive. To look at the distribution of neurons with different neurite lengths in the population, we plotted the percentage of neurons according to the neurite length (FIG. 6F). The curves for neurons plated on CPG15, CPG 15-2, and both proteins together all overlapped, indicating that the two proteins affect a similar subpopulation of cortical neurons to a similar degree.

In summary, CPG 15 and CPG 15-2 enhanced initial neurite outgrowth as well as neurite extension in dissociated cortical neurons. The efficacies of the two proteins were indistinguishable for all the parameters examined and there was no additive effect with joint application.

### Example 8

### CPG15 and CPG15-2 Promote Survival of Cortical Neurons

Another known function of CPG 15 is promoting neuronal survival by preventing starvation-induced apoptosis. To test the survival effect of CPG15-2 and to directly compare the results with the growth-promoting effects, we differentially stained live and dead cells in the dissociated cultures we used for the neurite growth assay. For survival assays, the same cultures at 24 hours after plating were stained with Live/dead viability/cytotoxicity kit (Molecular Probes, Eugene, Oreg.), and the number of live and dead cells were counted. Statistical significance was determined by ANOVA and SNK post hoc analysis using StatView software (SAS Institute).

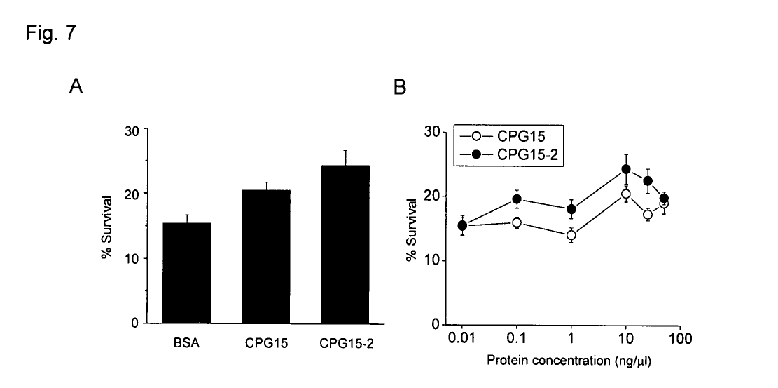

Neurons plated on CPG15 or CPG15-2 coated dishes showed a higher survival rate compared to those plated on BSA coated dishes (FIGS. 7A and 7B), indicating that both proteins have a survival promoting effect. The two proteins showed similar effects at similar concentrations, indicating that both proteins have similar efficacy as in the case of neurite growth effects.

### Example 9

### Starvation and Apoptosis Assay Protocols

As shown in FIG. 8, primary hippocampal or cortical neurons were plated essentially as described in Zhou et al. (FEBS Letters 526:21-25, 2002). Briefly, E18 Sprague-Dawley rat embryos were collected in ice-cold Hank's buffered salt solution (HBSS, Sigma). Cortices were dissected out and digested for 15 minutes at 37° C. in 0.25% trypsin (Gibco) and 0.1% DNase (Sigma) in HBSS. After digestion, the tissue was washed three times in ice-cold HBSS and then triturated with fire-polished Pasteur pipettes of decreasing pore size in HBSS with 0.1% DNase. After centrifugation for 10 minutes at 1,000 rpm, the cell pellet was resuspended in plating media consisting of Neurobasal medium (Gibco) supplemented with B27 (Gibco), L-glutamine (500 μM), and L-glutamate (25 μM). Cells were then counted and plated at 0.75×10⁵ cells/well in twelve well plates, each well containing one 10 mm glass coverslip (Assistent) that had been preincubated overnight in 40 μg/ml poly-D-lysine (Fischer) and 2.5 μg/ml laminin (Fischer), rinsed three times in water, and then incubated in plating medium. After four days in vitro (DIV), half the plating media was replaced with feeding medium (plating medium minus L-glutamate). Cultures were maintained in a humidified 37° C. incubator with a 5% CO₂ atmosphere.

After 6 DIV, cortical neurons were washed three times with Neurobasal medium without supplements, then incubated for 12 hours in the unsupplemented medium with or without 5 μg/ml purified CPG15-2 protein. After an additional 12 hours in feeding medium, cells were fixed in 4% formaldehyde/PBS for 30 minutes at 4° C. before Hoechst staining or immunocytochemistry. Fixed cells were incubated 30 minutes with Hoechst 33342 (1:1000, in PBS, Sigma), rinsed three times in PBS and mounted onto slides with Fluoromount G (Southern Biotechnology). For immunocytochemistry, fixed cells were washed with PBS for five minutes, then permeabilized with 0.3% Triton X-100 for five minutes at 4° C. Neurons were washed again with PBS, incubated with blocking solution (10% goat serum, 0.1% Triton X-100 in PBS) for one hour at 4° C., and then incubated with an anti-cleaved Caspase3 antibody (1:100, Cell Signaling Technology) in blocking solution overnight at 4° C. After rinsing three times with PBS, an anti-rabbit secondary antibody coupled to rhodamine (1:500, Jackson Immuno Research) was added for one hour at room temperature. Chromatin staining with Hoechst was done simultaneously, and neurons were rinsed and mounted as described above.

For quantification, fragmented apoptotic nuclei as well as healthy nuclei were counted blind to experimental treatment using a fluorescence microscope with UV filter setting for the Hoechst staining (excitation 330-380; emission 420) and rhodamine settings for visualizing the antibody against cleaved caspase3 (excitation 528-553; emission 600-660). Treatments were repeated in three independent experiments with two coverslips per treatment in each experiment. Each data point represents the mean of 500-600 cells, counted in 40-50 different fields per coverslip. The percent apoptotic cells was calculated based on the number of condensed/fragmented nuclei divided by the total number of nuclei. Comparisons between groups were analyzed using a student's t-test.

### OTHER EMBODIMENTS

From the foregoing description, one skilled in the art can easily ascertain the essential characteristics of this invention; can make various changes and modifications of the invention to adapt it to various usages and conditions. Thus, other embodiments are also within the claims.

All publications and patents mentioned in this specification are herein incorporated by reference to the same extent as if each individual publication or patent was specifically and individually incorporated by reference. In addition, U.S. Patent Publication Number 2004 176291 and PCT Publication Number WO2004/031347 are herein incorporated by reference in their entirety.

What is claimed is:

1. A method of treating or preventing a condition of excessive cell death in a subject, said method comprising administering to said subject a CPG15-2, wherein said CPG-15 comprises a sequence with at least 85% sequence identity to SEQ ID NO: 2 in an amount and for a time sufficient to prevent, reduce, or eliminate at least one of the symptoms of said condition.

2. The method of claim 1, wherein said CPG15-2 is a membrane bound CPG15-2.

3. The method of claim 1, wherein said CPG15-2 is a soluble CPG15-2.

4. The method of claim 1, wherein said CPG15-2 comprises a sequence substantially identical to the sequence of SEQ ID NOS: 2, 6, or 20.

5. The method of claim 1, wherein said CPG15-2 comprises a post-translational modification.

6. The method of claim 5, wherein said post-translational modification comprises glycosylation of at least one amino acid residue.

7. The method of claim 5, wherein said post-translational modification comprises the attachment of a membrane component to said CPG15-2.

8. The method of claim 1, wherein said cell death is mediated by apoptosis.

9. The method of claim 1, wherein said at least one symptom of said condition is reduced by at least 5% relative to a control subject not treated by said method.

10. The method of claim 1, wherein said condition is a condition selected from the group consisting of neurological conditions; conditions of the retina and optic nerve; conditions of the bone, skin, muscle, joint, or cartilage; cardiac conditions, autoimmune conditions, liver conditions; ischemia; aging or an aging related condition; and immunodeficiency conditions.

11. The method of claim 10, wherein said neurological condition is Alzheimer's Disease, Parkinson's Disease, Huntington's Disease, Amyotrophic Lateral Sclerosis, traumatic injury to the brain, or stroke.

12. The method of claim 10, wherein said cardiac condition is cardiac ischemia.

13. The method of claim 10, wherein said condition of the retina and optic nerve is retinitis pigmentosa.

14. The method of claim 10, wherein said condition of the retina and optic nerve is macular degeneration.

15. A method of reducing or preventing cell death, said method comprising administering to a cell a CPG 15-2, wherein said CPG15-2 comprises a sequence with at least 85% sequence identity to SEQ ID NO: 2 in an amount and for a time sufficient to reduce or prevent said cell death.

16. The method of claim 15, wherein said cell is a cell predisposed to apoptosis.

17. The method of claim 15, wherein said cell death is reduced by at least 15% as compared to a cell not treated by said method.

18. The method of claim 15, wherein said cell is selected from the group consisting of a cell of the nervous system, muscle cell, stem cell, immune cell, blood cell, endothelial cell, fibroblast cell, epithelial cell, bone cell, skin cell, pancreatic cell, liver cell, cardiomyocyte, oligodendrocyte, and chondrocyte.

19. The method of claim 18, wherein said cell of the nervous system is a central nervous system neuron, a peripheral nervous system neuron, or a spinal cord neuron.

20. The method of claim 15, wherein said CPG15-2 comprises a sequence substantially identical to the sequence of SEQ ID NOS: 2, 6, or 20.

21. The method of claim 15, wherein said CPG15-2 comprises a post-translational modification.

22. A method of promoting the survival or differentiation of a cell comprising administering to said cell a CPG 15-2, wherein said CPG 15-2 comprises a sequence with at least 85% sequence identity to SEQ ID NO: 2 for a time and in an amount to promote the survival or differentiation of said cell.

23. The method of claim 22, wherein said cell is selected from the group consisting of a cell of the nervous system, muscle cell, stem cell, immune cell, blood cell, endothelial cell, fibroblast cell, epithelial cell, bone cell, skin cell, pancreatic cell, liver cell, cardiomyocyte, oligodendrocyte, and chondrocyte.

24. The method of claim 22, wherein said CPG 15-2 comprises a sequence substantially identical to the sequence of SEQ ID NOS: 2, 6, or 20.

25. The method of claim 22, wherein said CPG15-2 comprises a post-translational modification.

26. The method of claim 22, wherein said cell is a tissue culture cell.

27. The method of claim 26, wherein said CPG15-2 is added to the culture media of said tissue culture cell.

28. The method of claim 22, wherein said survival or differentiation of said cell is increased by at least 15% as compared to a control cell not treated by said method.


SEQUENCE LISTING (21 sequences)

SEQ ID NO: 1 - DNA, 705 bp, Homo sapiens
acccccagctcccatccaatcaccgtccctccctccgggggctgggcggacgggctgcactccaagcagctagctcctgcactaggctctcagccagggatgatgcgctgctgccgccgccgctgctgctgccggcaaccaccccatgccctgaggccgttgctgttgctgcccctcgtccttttacctcccctggcagcagctgcagcgggcccaaaccgatgtgacaccatataccagggcttcgccgagtgtctcatccgcttgggggacagcatgggccgcggaggcgagctggagaccatctgcaggtcttggaatgacttccatgcctgtgcctctcaggtcctgtcaggctgtccggaggaggcagctgcagtgtgggaatcactacagcaagaagctcgccaggccccccgtccgaataacttgcacactctgtgcggtgccccggtgcatgttcgggagcgcggcacaggctccgaaaccaaccaggagacgctgcgggctacagcgcctgcactccccatggcccctgcgcccccactgctggcggctgctctggctctggcctacctcctgaggcctctggcctagcttgttgggttgggtagcagcgcccgtacctccagccctgctctggcggtggttgtccaggctctgcagagcgcagcagggcttttcattaaaggtatttatatttgt

SEQ ID NO: 2 - protein, 165 aa, Homo sapiens
MMRCCRRRCCCRQPPHALRPLLLLPLVLLPPLAAAAAGPNRCDTIYQGFAECLIRLGDSMGRGGELETICRSWNDFHACASQVLSGCPEEAAAVWESLQQEARQAPRPNNLHTLCGAPVHVRERGTGSETNQETLRATAPALPMAPAPPLLAAALALAYLLRPLA

SEQ ID NO: 3 - DNA, 608 bp, Mus musculus
gacagccagggatgatgtgcaactgctgccactgccactggcgccgccgctgtcagcggctaccctgtgccctgacgctgttgctgctactaccactcgcagtggcctctgagggcccaaaccgctgtgataccatataccaaggctttgctgaatgtctcatccgcctgggggatggcatgggtcgaggaggcgagctacagactgtctgcagatcctggaatgacttccacgcctgtgcctctcgggtcctgtcaggctgcccagaggaggcggctgcagtgtgggagtcactgcagcaagaagctcgccgcgccccacacccagataatttgcacatcctctgtggcgctcctgtgagtgttcgggagcggattgctggcccagagaccaaccaggagacactacgggccacagctcctgcactggctccagctccagcccctgtgttgctcgccgccgctctagcgcttgcctgcctcctggggcctctggcctaaacagtctggttggccagccaacagtgcccttgcctcccatcactgcatgcagtggctgccatgtgagctctgcagtatgcacacttttcattaaaggtatttatattc

SEQ ID NO: 4 - protein, 162 aa, Mus musculus
MMCNCCHCHWRRRCQRLPCALTLLLLLPLAVASEGPNRCDTIYQGFAECLIRLGDGMGRGGELQTVCRSWNDFHACASRVLSGCPEEAAAVWESLQQEARRAPHPDNLHILCGAPVSVRERIAGPETNQETLRATAPALAPAPAPVLLAAALALACLLGPLA

SEQ ID NO: 5 - protein, 104 aa, Mus musculus
SEGPNRCDTIYQGFAECLIRLGDGMGRGGELQTVCRSWNDFHACASRVLSGCPEEAAAVWESLQQEARRAPHPDNLHILCGAPVSVRERIAGPETNQETLRATA

SEQ ID NO: 6 - protein, 104 aa, Homo sapiens
AAGPNRCDTIYQGFAECLIRLGDSMGRGGELETICRSWNDFHACASQVLSGCPEEAAAVWESLQQEARQAPRPNNLHTLCGAPVHVRERGTGSETNQETLRATA

SEQ ID NO: 7 - DNA, 34 bp, Artificial Sequence
  Primer
ccgctcgagccaccatgatgtgcaactgctgcca

SEQ ID NO: 8 - DNA, 28 bp, Artificial Sequence
  Primer
tccccgcggttaggccagaggccccagg

SEQ ID NO: 9 - DNA, 30 bp, Artificial Sequence
  Primer
actctctcactctctttctgtctcttcctc

SEQ ID NO: 10 - DNA, 30 bp, Artificial Sequence
  Primer
acagtctgaaaagcccttaaagactgcatc

SEQ ID NO: 11 - DNA, 24 bp, Artificial Sequence
  Primer
ttgtaaccaactgggacgatatgg

SEQ ID NO: 12 - DNA, 24 bp, Artificial Sequence
  Primer
gatcttgatcttcatggtgctagg

SEQ ID NO: 13 - DNA, 33 bp, Artificial Sequence
  Primer
ggaattcgccaccatgggacttaagttgaacgg

SEQ ID NO: 14 - DNA, 25 bp, Artificial Sequence
  Primer
ggggtaccgcctgctgctctcacgg

SEQ ID NO: 15 - DNA, 46 bp, Artificial Sequence
  Primer
ggggtacccatcaccatcaccatcacaagtgcgatgcagtctttaa

SEQ ID NO: 16 - DNA, 26 bp, Artificial Sequence
  Primer
ggggtaccgtttgggccctcagaggc

SEQ ID NO: 17 - DNA, 53 bp, Artificial Sequence
  Primer
ggggtaccgactataaggacgatgatgacaagcgctgtgataccatataccaa

SEQ ID NO: 18 - DNA, 53 bp, Artificial Sequence
  Primer
ggggtacccatcaccatcaccatcacgcaggccgctgtgataccatataccaa

SEQ ID NO: 19 - protein, 136 aa, Mus musculus
MMCNCCHCHWRRRCQRLPCALTLLLLLPLAVASEGPNRCDTIYQGFAECLIRLGDGMGRGGELQTVCRSWNDFHACASRVLSGCPEEAAAVWESLQQEARRAPHPDNLHILCGAPVSVRERIAGPETNQETLRATA

SEQ ID NO: 20 - protein, 139 aa, Homo sapiens
MMRCCRRRCCCRQPPHALRPLLLLPLVLLPPLAAAAAGPNRCDTIYQGFAECLIRLGDSMGRGGELETICRSWNDFHACASQVLSGCPEEAAAVWESLQQEARQAPRPNNLHTLCGAPVHVRERGTGSETNQETLRATA

SEQ ID NO: 21 - protein, 142 aa, Mus musculus
MGLKLNGRYISLILAVQIAYLVQAVRAAGKCDAVFKGFSDCLLKLGDSMANYPQGLDDKTNIKTVCTYWEDFHSCTVTALTDCQEGAKDMWDKLRKESKNLNIQGSLFELCGSSNGAAGSLLPALSVLLVSLSAALATWFSF